(19) United States
(12) Patent Application Publication
(10) Pub. No.: US 2022/0396614 A1
(43) Pub. Date: Dec. 15, 2022

(54) PLASMODIUM ANTIBODIES

(71) Applicants: Deutsches Krebsforschungszentrum, Heidelberg (DE); Max Planck Institute for Infection Biology, Berlin (DE); SickKids, Toronto (CA); Universitätsklinikurn Tübingen Institut für Tropenmedizin, Tübingen (DE)

(72) Inventors: Hedda Wardemann, Heidelberg (DE); Rajagopal Murugan, Heidelberg (DE); Tizian Decker, Heidelberg (DE); Eiena Levashina, Berlin (DE); Giulia Costa, Berlin (DE); Jean-Philipp Julien, Toronto (CA); Stephen Scally, Toronto (CA); Elaine Thai, Toronto (CA); Beniamin Mordmüller, Tübingen (DE)

(21) Appl. No.: 17/624,046

(22) PCT Filed: Jun. 30, 2020
(86) PCT No.: PCT/EP2020/068359

§ 371 Date: Dec. 30, 2021

(30) Foreign Application Priority Data
Jul. 1, 2019 (EP) 19183681.6

(51) Int. Cl. C07K 16/20; C12Q 1/02; A61P 33/06

(52) U.S. Cl. CPC C07K 16/205; C12Q 1/02; A61P 33/06; C07K 2317/34; C07K 2317/92; C07K 2317/76; C07K 2317/24

(57) ABSTRACT

The present invention relates to an antibody binding to a peptide comprising an amino acid sequence NANP (SEQ ID NO:1) and to at least one peptide comprising an amino acid sequence selected from NVDP (SEQ ID NO:2), NPDP (SEQ ID NO:3), and KQPA (SEQ ID NO:4), to a polynucleotide or polynucleotides encoding said antibody, and to said antibody for use in medicine and for use in prevention of Plasmodium infection. Moreover, the present invention relates to methods, kits, and devices related thereto.

The present invention relates to an antibody binding to a peptide comprising an amino acid sequence NANP (SEQ ID NO:1) and to at least one peptide comprising an amino acid sequence selected from NVDP (SEQ ID NO:2), NPDP (SEQ ID NO:3), and KQPA (SEQ ID NO:4), to a polynucleotide or polynucleotides encoding said antibody, and to said antibody for use in medicine and for use in prevention of Plasmodium infection. Moreover, the present invention relates to methods, kits, and devices related thereto.

Plasmodium falciparum (Pf) is a unicellular apicomplexan parasite that causes malaria, a life-threatening vector-borne disease. Pf sporozoites, the parasite stage that is transmitted to humans by infectious Anopheles mosquitoes, are densely covered by circumsporozoite protein (PfCSP). PfCSP plays a key role in parasite development in the mosquito vector and establishment of the infection in the human host. It consists of an amino (N) terminal domain, a highly disordered central region made up of only four amino acids (asparagine (N), alanine (A), valine (V), proline (P)) arranged in NANP repeat motifs, as well as a carboxy (C) terminal domain that anchors the protein to the sporozoite surface by a GPI anchor. In contrast to the C-terminus with substantial sequence diversity, the NANP motif is fully conserved in all Pf parasites isolated so far and shows variability only in the number of repeats. The central NANP domain also represents the major PfCSP B cell epitope on sporozoites and induces dominant serum antibody responses. Based on historic observations that antibodies against the central repeat inhibit Pf in vitro and can protect from Plasmodium infection in animal models, a PfCSP-based malaria vaccine (RTS,S) has been developed. RTS,S contains a truncated version of PfCSP composed of 18.5 NANP repeats and the PfCSP C-terminal domain. To increase immunogenicity, the truncated PfCSP protein is fused to Hepatitis B surface antigen (HBsAg) and further complexed into particles by co-expression with free HBsAg. A large pediatric Phase III clinical trial with RTS,S in AS01, a potent liposome-based adjuvant, has been performed at eleven malaria endemic sites in seven African countries. Although RTS,S-induced anti-NANP antibody titers correlated with clinical protection, the overall efficacy of the vaccine even after a booster immunization was below 40% within two-years and therefore needs to be improved. A handful of studies have recently applied antibody-cloning strategies to isolate protective human antibodies against PfCSP and delineated their target epitopes as basis for the development of a second-generation PfCSP-based malaria vaccine (Triller et al., Immunity 47, 1197-1209.e10 (2017); Tan, et al., Nat. Med. 24, 401-407 (2018)).

There is, thus, still a need in the art to provide reliable means to improve prevention of Plasmodium infections. In particular, there is a need to provide means and methods avoiding at least in part the drawbacks of the prior art as discussed above.

This problem is solved by the methods, compositions, and uses with the features of the independent claims. Preferred embodiments, which might be realized in an isolated fashion or in any arbitrary combination are listed in the dependent claims.

Accordingly, the present invention relates to an antibody binding to a peptide comprising an amino acid sequence NANP (SEQ ID NO:1) and to at least one peptide comprising an amino acid sequence selected from NVDP (SEQ ID NO:2), NPDP (SEQ ID NO:3), and KQPA (SEQ ID NO:4).

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

As used herein, the term “standard conditions”, if not otherwise noted, relates to IUPAC standard ambient temperature and pressure (SATP) conditions, i.e. preferably, a temperature of 25° C. and an absolute pressure of 100 kPa; also preferably, standard conditions include a pH of 7. Moreover, if not otherwise indicated, the term “about” relates to the indicated value with the commonly accepted technical precision in the relevant field, preferably relates to the indicated value±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1%, most preferably less than 0.1% by weight of non-specified component(s). In the context of nucleic acid sequences, the term “essentially identical” indicates a % identity value of at least 80%, preferably at least 90%, more preferably at least 98%, most preferably at least 99%. As will be understood, the term essentially identical includes 100% identity. The aforesaid applies to the term “essentially complementary” mutatis mutandis.

The term “antibody”, as used herein, refers to all types of antibodies having the activity of, preferably specifically, binding to a peptide comprising an epitope having the amino acid sequence NANP (SEQ ID NO:1) and to at least one peptide comprising an epitope having an amino acid sequence selected from NVDP (SEQ ID NO:2), NPDP (SEQ ID NO:3), and KQPA (SEQ ID NO:4). Epitopes as referred to herein are, preferably, defined by stretches of 4 to 15, preferably 4 to 11, more preferably 4, amino acids in length, which may be continuous or non-continuous. Thus, preferably, the epitope may be a conformational epitope; more preferably, the amino acids are continuous. Thus, more preferably, an epitope is a continuous amino acid sequence in a polypeptide comprising, preferably consisting of, at least one of SEQ ID NOs:1 to 4. Specific binding in this context means that the antibody of the invention essentially binds to at least two of the aforesaid epitopes without significant cross-reactivity (i.e. binding) to other epitopes. Specific binding can be determined by techniques well known in the art; preferably, specific binding is binding of the antibody with a dissociation constant K_{D} at least 10-fold, preferably at least 100-fold, more preferably at least 1000-fold lower for a peptide comprising the amino acid sequence NANP and for least one peptide comprising an amino acid sequence selected from NVDP, NPDP, and KQPA, compared to any other peptide. Preferably, the antibody is a mammalian antibody, more preferably is a human or humanized, mouse, rat, rabbit, goat, guinea pig, donkey, or horse antibody, more preferably is a human or a humanized antibody, most preferably is a human antibody. The antibody preferably is an IgG, IgM, IgA, IgD, or IgE, preferably is an IgG. The antibody may be comprised in a polyclonal serum, or may be enriched or partially or fully purified, e.g. by affinity chromatography. Preferably, the antibody is a monoclonal antibody, a single chain antibody, a chimeric antibody, a nanobody, or any fragment or derivative of such antibody having the above mentioned binding properties. Preferred fragments and derivatives comprised by the term antibody as used herein encompass a synthetic antibody, an Fab, F(ab)₂ Fv or scFv fragment, and a chemically modified derivative of any of these antibodies. Chemical modifications envisaged preferably by the present invention include those which aim to couple the antibody to a detectable marker as specified elsewhere in this specification, in particular to a dye, or to modifying plasma half-life of the antibody, e.g. PEGylation. An also preferred variant is a variant comprising a stabilizing mutation. Antibodies or fragments thereof, in general, can be obtained by using methods which are described, e.g., in Harlow and Lane “Antibodies, A Laboratory Manual”, CSH Press, Cold Spring Harbor, 1988.

The antibody has the activity of binding to a peptide comprising an amino acid sequence NANP (SEQ ID NO:1) and to at least one peptide comprising an amino acid sequence selected from NVDP (SEQ ID NO:2), NPDP (SEQ ID NO:3), and KQPA (SEQ ID NO:4). Thus, the antibody binds, preferably specifically, to the epitopes NANP and NVDP; or binds, preferably specifically, to the epitopes NANP and NPDP; or binds, preferably specifically, to the epitopes NANP and KQPA; or binds, preferably specifically, to the epitopes NANP, NVDP, and NPDP; or binds, preferably specifically, to the epitopes NANP, NVDP, and KQPA; or binds, preferably specifically, to the epitopes NANP, NPDP, and KQPA; or binds, preferably specifically, to the epitopes NANP, NVDP, NPDP, and KQPA. Preferably, the antibody binds, preferably specifically, to the epitopes NANP and NVDP. More preferably, the antibody binds, preferably specifically, to the epitopes NANP, NVDP, and NPDP. Most preferably, the antibody binds, preferably specifically, to the epitopes NANP, NVDP, NPDP, and KQPA. The binding affinity of the antibody to the aforesaid epitopes need not be the same for all epitopes bound; however, preferably, the dissociation constant K_{D} for the antibody and the respective epitope is at most 10⁻⁵ M, preferably at most 5×10⁻⁶ M, more preferably at most 2×10⁻⁶ M, most preferably at most 10⁻⁷ M, in particular for the KQPA. More preferably, the dissociation constant K_{D} for the antibody and the respective epitope is at most 10⁻⁶ M, preferably at most 2×10⁻⁷ M, more preferably at most 10⁻⁷ M, even more preferably at most 5×10⁻⁸ M, still more preferably at most 2×10⁻⁸ M, most preferably at most 10⁻⁸ M, in particular for epitopes NANP, NVDP, and NPDP. Preferably, the peptide comprising the amino acid sequence NANP comprises, more preferably consists of, the amino acid sequence NPNANPNANPNANPNANPNANP (SEQ ID NO:44) or NANPNANPNANPNANPNANP (SEQ ID NO:45). Preferably, the peptide comprising the amino acid sequence KQPA comprises, more preferably consists of, the amino acid sequence KQPADGNPDPNANPN (SEQ ID NO:37). Also preferably, the peptide comprising the amino acid sequence NPDP comprises, more preferably consists of, the amino acid sequence NPDPNANPNVDPNANP (SEQ ID NO:38). Also preferably, the peptide comprising the amino acid sequence NVDP comprises, more preferably consists of, the amino acid sequence NVDPNANPNVDPNANPNVDP (SEQ ID NO:39).

Preferably, the antibody comprises complementarity determining regions (CDRs) comprising the sequences of SEQ ID NOs:5 to 10 or SEQ ID NOs:11 to 16. More preferably, the antibody is a monoclonal antibody (mAb) 4493 as shown herein in the Examples and comprises a heavy chain comprising a CDR1 having an amino acid sequence GFTFGDYA (SEQ ID NO:5), a CDR2 having an amino acid sequence IRSKANGGRT (SEQ ID NO:6), and a CDR3 having an amino acid sequence TRVELGSSWSLGY (SEQ ID NO:7); and comprises a light chain comprising a CDR1 having an amino acid sequence QSVSSTY (SEQ ID NO:8), a CDR2 having an amino acid sequence GAS (SEQ ID NO:9), and a CDR3 having an amino acid sequence QQYGSSPWT (SEQ ID NO:10). Preferably, the heavy chain CDRs1-3 are encoded by a polynucleotide comprising the sequences of SEQ ID NOs:17 to 19, and the light chain CDRs1-3 are encoded by a polynucleotide comprising the sequences of SEQ ID NOs:20 to 22. Also preferably, the antibody is monoclonal antibody 2541 shown in herein the Examples and preferably comprises a heavy chain comprising a CDR1 having an amino acid sequence GFTFSSYG (SEQ ID NO:11), a CDR2 having an amino acid sequence IWHDGSKK (SEQ ID NO:2), and a CDR3 having an amino acid sequence ARVGDYSDFKYGAFDI (SEQ ID NO:13); and comprises a light chain comprising a CDR1 having an amino acid sequence QSISSW (SEQ ID NO:14), a CDR2 having an amino acid sequence KAS (SEQ ID NO:15), and a CDR3 having an amino acid sequence QQYNSYWT (SEQ ID NO:16). Preferably, the heavy chain CDRs1-3 are encoded by a polynucleotide comprising the sequences of SEQ ID NOs:23 to 25, and the light chain CDRs1-3 are encoded by a polynucleotide comprising the sequences of SEQ ID NOs:26 to 28. Preferably, CDRs are annotated according to standards of the International Immunogenetics Database (www.imgt.org) as of Jun. 30, 2019.

More preferably, the heavy chain of mAB4493 has the amino acid sequence of SEQ ID NO:29 or a sequence at least 50%, preferably at least 70%, more preferably at least 80%, still more preferably at least 90%, still more preferably at least 95%, most preferably at least 99%, identical to SEQ ID NO:29; still more preferably, the heavy chain of mAB4493 has the amino acid sequence of SEQ ID NO: 29. Preferably, the heavy chain of the antibody comprises an arginine at a position corresponding to position 52 of SEQ ID NO:29; more preferably is encoded by the immunoglobulin heavy variable 3-49 gene (IGHV3-49). Also more preferably, the light chain of mAB4493 has the amino acid sequence of SEQ ID NO: 30 or a sequence at least 50%, preferably at least 70%, more preferably at least 80%, still more preferably at least 90%, still more preferably at least 95%, most preferably at least 99%, identical to SEQ ID NO:30; more preferably, the light chain of mAB4493 has the amino acid sequence of SEQ ID NO: 30. Also more preferably, the heavy chain of mAB2541 has the amino acid sequence of SEQ ID NO:31 or a sequence at least 50%, preferably at least 70%, more preferably at least 80%, still more preferably at least 90%, still more preferably at least 95%, most preferably at least 99%, identical to SEQ ID NO:31; still more preferably, the heavy chain of mAB2541 has the amino acid sequence of SEQ ID NO: 31. Also more preferably, the light chain of mAB2541 has the amino acid sequence of SEQ ID NO: 32 or a sequence at least 50%, preferably at least 70%, more preferably at least 80%, still more preferably at least 90%, still more preferably at least 95%, most preferably at least 99%, identical to SEQ ID NO:32; more preferably, the light chain of mAB2541 has the amino acid sequence of SEQ ID NO: 32.

Preferably, variants of the aforesaid antibodies are also included. As used herein, the term “antibody variant” relates to any chemical molecule comprising at least one antibody as specified elsewhere herein, having the indicated activity, but differing in primary structure from said antibody. Thus, the antibody variant, preferably, is a mutein having the indicated biological activity. Preferably, the antibody variant comprises a peptide having an amino acid sequence corresponding to an amino acid sequence of 5 to 1000, more preferably 50 to 900, most preferably, 100 to 800 consecutive amino acids comprised in an antibody as specified above. Moreover, it is to be understood that an antibody variant as referred to in accordance with the present invention shall preferably have an amino acid sequence which differs due to at least one amino acid substitution, deletion and/or addition, wherein the amino acid sequence of the variant is still, preferably, at least 70%, 80%, 85%, 90%, 92%, 95%, 97%, 98%, or 99% identical with the amino acid sequence of the specific antibody. The degree of identity between two amino acid sequences can be determined by algorithms well known in the art. Preferably, the degree of identity is to be determined by comparing two optimally aligned sequences over a comparison window, where the fragment of amino acid sequence in the comparison window may comprise additions or deletions (e.g., gaps or overhangs) as compared to the sequence it is compared to for optimal alignment. The percentage is calculated by determining, preferably over the whole length of the polypeptide, the number of positions at which the identical amino acid residue occurs in both sequences to yield the number of matched positions, dividing the number of matched positions by the total number of positions in the window of comparison and multiplying the result by 100 to yield the percentage of sequence identity. Preferably, the comparison window comprises a complete sequence as specified herein. Optimal alignment of sequences for comparison may be conducted by the local homology algorithm of Smith and Waterman (1981), by the homology alignment algorithm of Needleman and Wunsch (1970), by the search for similarity method of Pearson and Lipman (1988), by computerized implementations of these algorithms (GAP, BESTFIT, BLAST, PASTA, and TFASTA in the Wisconsin Genetics Software Package, Genetics Computer Group (GCG), 575 Science Dr., Madison, Wis.), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. Antibody variants referred to herein may be allelic variants or any other species specific homologs, paralogs, or orthologs. Moreover, the antibody variants referred to herein preferably include fragments of the specific antibodies as specified herein above. Such fragments may be or be derived from, e.g., degradation products or splice variants of the polypeptides. Further included are variants which differ due to posttranslational modifications such as phosphorylation, glycosylation, ubiquitinylation, sumoylation, or myristylation, by including non-natural amino acids, and/or by being peptidomimetics.

Advantageously, it was found in the work underlying the present invention that antibodies recognizing more than one epitope from a Plasmodium circumsporozoite protein provide for better inhibition of circumsporozoite infection than monospecific antibodies. Monoclonal antibodies 4493 and 2541 were found to be particularly advantageous.

The definitions made above apply mutatis mutandis to the following. Additional definitions and explanations made further below also apply for all embodiments described in this specification mutatis mutandis.

The present invention further relates to a polynucleotide or polynucleotides encoding the antibody according to the present invention.

The term “polynucleotide” is known to the skilled person. As used herein, the term includes nucleic acid molecules comprising or consisting of a nucleic acid sequence or nucleic acid sequences as specified herein. As will be understood, the heavy chain and the light chain of an antibody may be encoded by a single polynucleotide, or may be encoded by two separate polynucleotides, which may e.g. be provided as a kit. The polynucleotide or polynucleotides of the present invention shall be provided, preferably, either as an isolated polynucleotide or polynucleotides (i.e. isolated from the natural context) or in genetically modified form. The polynucleotide or polynucleotides, preferably, is DNA, including cDNA, or is RNA. The term encompasses single as well as double stranded polynucleotides. Preferably, the polynucleotide is a chimeric molecule, i.e., preferably, comprises at least one nucleic acid sequence, preferably of at least 20 bp, more preferably at least 100 bp, heterologous to the residual nucleic acid sequences. Moreover, preferably, comprised are also chemically modified polynucleotides including naturally occurring modified polynucleotides such as glycosylated or methylated polynucleotides or artificial modified one such as biotinylated polynucleotides.

Preferably, the heavy chain of mAB4493 is encoded by a polynucleotide comprising the nucleic acid sequence of SEQ ID NO: 33 or a sequence at least 50%, preferably at least 70%, more preferably at least 80%, still more preferably at least 90%, still more preferably at least 95%, most preferably at least 99%, identical to SEQ ID NO:33; still more preferably, the heavy chain of mAB4493 is encoded by a polynucleotide comprising the nucleic acid sequence of SEQ ID NO: 33. Also more preferably, the light chain of mAB4493 is encoded by a polynucleotide comprising the nucleic acid sequence of SEQ ID NO: 34 or a sequence at least 50%, preferably at least 70%, more preferably at least 80%, still more preferably at least 90%, still more preferably at least 95%, most preferably at least 99%, identical to SEQ ID NO:34; more preferably, the light chain of mAB4493 is encoded by a polynucleotide comprising the nucleic acid sequence of SEQ ID NO: 34. Also more preferably, the heavy chain of mAB2541 is encoded by a polynucleotide comprising the nucleic acid sequence of SEQ ID NO: 35 or a sequence at least 50%, preferably at least 70%, more preferably at least 80%, still more preferably at least 90%, still more preferably at least 95%, most preferably at least 99%, identical to SEQ ID NO:35; still more preferably, the heavy chain of mAB2541 is encoded by a polynucleotide comprising the nucleic acid sequence of SEQ ID NO:35. Also more preferably, the light chain of mAB2541 is encoded by a polynucleotide comprising the nucleic acid sequence of SEQ ID NO:36 or a sequence at least 50%, preferably at least 70%, more preferably at least 80%, still more preferably at least 90%, still more preferably at least 95%, most preferably at least 99%, identical to SEQ ID NO:36; more preferably, the light chain of mAB4493 is encoded by a polynucleotide comprising the nucleic acid sequence of SEQ ID NO:36.

As used herein, the term polynucleotide, preferably, includes variants of the specifically indicated polynucleotides. More preferably, the term polynucleotide relates to the specific polynucleotides indicated. It is to be understood, however, that a polypeptide having a specific amino acid sequence may be encoded by a variety of polynucleotides, due to the degeneration of the genetic code. The skilled person knows how to select a polynucleotide encoding a polypeptide having a specific amino acid sequence and also knows how to optimize the codons used in the polynucleotide according to the codon usage of the organism used for expressing said polynucleotide. Thus, the term “polynucleotide variant”, as used herein, relates to a variant of a polynucleotide related to herein comprising a nucleic acid sequence characterized in that the sequence can be derived from the aforementioned specific nucleic acid sequence by at least one nucleotide substitution, addition and/or deletion, wherein the polynucleotide variant shall have the activity as specified for the specific polynucleotide, i.e. shall encode at least one chain of an antibody according to the present invention. Preferably, said polynucleotide variant is an ortholog, a paralog or another homolog of the specific polynucleotide. Also preferably, said polynucleotide variant is a naturally occurring allele of the specific polynucleotide. Polynucleotide variants also encompass polynucleotides comprising a nucleic acid sequence, which is capable of hybridizing to the aforementioned specific polynucleotides, preferably, under stringent hybridization conditions. These stringent conditions are known to the skilled worker and can be found in Current Protocols in Molecular Biology, John Wiley & Sons, N. Y. (1989), 6.3.1-6.3.6. A preferred example for stringent hybridization conditions are hybridization conditions in 6× sodium chloride/sodium citrate (=SSC) at approximately 45° C., followed by one or more wash steps in 0.2×SSC, 0.1% SDS at 50 to 65° C. The skilled worker knows that these hybridization conditions differ depending on the type of nucleic acid and, for example when organic solvents are present, with regard to the temperature and concentration of the buffer. For example, under “standard hybridization conditions” the temperature differs depending on the type of nucleic acid between 42° C. and 58° C. in aqueous buffer with a concentration of 0.1× to 5×SSC (pH 7.2). If organic solvent is present in the abovementioned buffer, for example 50% formamide, the temperature under standard conditions is approximately 42° C. The hybridization conditions for DNA:DNA hybrids are preferably for example 0.1×SSC and 20° C. to 45° C., preferably between 30° C. and 45° C. The hybridization conditions for DNA:RNA hybrids are preferably, for example, 0.1×SSC and 30° C. to 55° C., preferably between 45° C. and 55° C. The abovementioned hybridization temperatures are determined for example for a nucleic acid with approximately 100 bp (=base pairs) in length and a G+C content of 50% in the absence of formamide. The skilled worker knows how to determine the hybridization conditions required by referring to textbooks such as the textbook mentioned above, or the following textbooks: Sambrook et al., “Molecular Cloning”, Cold Spring Harbor Laboratory, 1989; Hames and Higgins (Ed.) 1985, “Nucleic Acids Hybridization: A Practical Approach”, IRL Press at Oxford University Press, Oxford; Brown (Ed.) 1991, “Essential Molecular Biology: A Practical Approach”, IRL Press at Oxford University Press, Oxford. Alternatively, polynucleotide variants are obtainable by PCR-based techniques such as mixed oligonucleotide primer-based amplification of DNA, i.e. using degenerated primers against conserved domains of a polypeptide of the present invention. Conserved domains of a polypeptide, e.g. scaffold domains of an antibody, are known to the skilled person or may be identified by a sequence comparison of the nucleic acid sequence of the polynucleotide or the amino acid sequence of various antibodies. As a template, DNA or cDNA from viruses, bacteria, fungi, plants, or animals, preferably from a virus, may be used. Further, variants include polynucleotides comprising nucleic acid sequences which are at least 70%, at least 75%, at least 80%, at least 85%, at least 90%, at least 95%, at least 98% or at least 99% identical to the specifically indicated nucleic acid sequences. Moreover, also encompassed are polynucleotides which comprise nucleic acid sequences encoding amino acid sequences which are at least 70%, at least 75%, at least 80%, at least 85%, at least 90%, at least 95%, at least 98% or at least 99% identical to the amino acid sequences specifically indicated. The percent identity values are, preferably, calculated over the entire amino acid or nucleic acid sequence region. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp (J. Mol. Evolution., 25, 351-360, 1987, Higgins et al., CABIOS, 5 1989: 151-153) or the programs Gap and BestFit [Needleman and Wunsch (J. Mol. Biol. 48; 443-453 (1970)) and Smith and Waterman (Adv. Appl. Math. 2; 482-489 (1981))], which are part of the GCG software packet (Genetics Computer Group, 575 Science Drive, Madison, Wis., USA 53711 (1991)), are to be used. The sequence identity values recited above in percent (%) are to be determined, preferably, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments.

A polynucleotide comprising a fragment of any of the specifically indicated nucleic acid sequences is also encompassed as a variant polynucleotide of the present invention. The fragment shall still encode an antibody, which still has the activity as specified. Accordingly, the antibody encoded may comprise or consist of the domains of the antibody of the present invention conferring the said biological activity. A fragment as meant herein, preferably, comprises at least 50, at least 100, at least 250 or at least 500 consecutive nucleotides of any one of the specific nucleic acid sequences or encodes an amino acid sequence comprising at least 20, at least 30, at least 50, at least 80, at least 100 or at least 150 consecutive amino acids of any one of the specific amino acid sequences.

The polynucleotides of the present invention either consist, essentially consist of, or comprise the aforementioned nucleic acid sequences. Thus, they may contain further nucleic acid sequences as well. Specifically, the polynucleotides of the present invention may encode fusion proteins wherein one partner of the fusion protein is an immunogenic polypeptide being encoded by a nucleic acid sequence recited above. Such fusion proteins may comprise as additional part polypeptides for monitoring expression (e.g., green, yellow, blue or red fluorescent proteins, alkaline phosphatase and the like), so called “tags” which may serve as a detectable marker or as an auxiliary measure for purification purposes, and/or scaffold polypeptides such as thioredoxin, as described herein above. Tags for the different purposes are well known in the art and are described elsewhere herein.

Preferably, the polynucleotide or polynucleotides as specified herein above are comprised in expression constructs. The term “expression construct”, as used herein, relates to a polynucleotide operatively linked to at least one expression control sequence causing transcription of the nucleic acid sequence comprised in said polynucleotide to occur, preferably in eukaryotic cells or isolated fractions thereof, preferably into a translatable mRNA or into a viral genome. Methods for providing expression constructs are known to the skilled person, who is also aware that expression of a given construct may be context dependent and may in particular depend on the type of cell (i.e. prokaryotic, archeal, or eukaryotic cell, plant or animal cells, specific cells type, and the like). Preferably, the expression construct is a eukaryotic expression construct, more preferably a mammalian expression construct. Regulatory elements ensuring expression in eukaryotic cells, preferably mammalian cells, are well known in the art. They, preferably, comprise regulatory sequences ensuring initiation of transcription and, optionally, poly-A signals ensuring termination of transcription and stabilization of the transcript. Additional regulatory elements may include transcriptional as well as translational enhancers.

The present invention also relates to the antibody as specified herein for use in medicine; and to the antibody as specified herein for use in prevention of a Plasmodium infection, preferably a Plasmodium falciparum infection, preferably for use in prevention of malaria.

The terms “preventing” and “prevention” refer to retaining health with respect to the diseases or disorders referred to herein for a certain period of time in a subject. It will be understood that said period of time may be dependent on the amount of the drug compound, which has been administered and individual factors of the subject discussed elsewhere in this specification. It is to be understood that prevention may not be effective in all subjects treated with the compound according to the present invention. However, the term requires that, preferably, a statistically significant portion of subjects of a cohort or population are effectively prevented from suffering from a disease or disorder referred to herein or its accompanying symptoms. Preferably, a cohort or population of subjects is envisaged in this context, which normally, i.e. without preventive measures according to the present invention, would develop a disease or disorder as referred to herein. Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001. Preferably, the treatment shall be effective for at least 10%, at least 20% at least 50% at least 60%, at least 70%, at least 80%, or at least 90% of the subjects of a given cohort or population. Preferably, preventing Plasmodium infection does not relate to providing sterile immunity; thus, preferably, prevention in the context of Plasmodium infection and in particular malaria relates to prevention of migration of sporozoites from the skin into the blood stream and/or infection of liver cells by the infectious agent; thus, more preferably, preventing Plasmodium infection is preventing infection to progress further and preventing the symptoms of malaria from occurring. Preferably, said preventing further comprises administration of anti-malarial drugs against blood stage parasites known to the skilled person, of vaccines or monoclonal antibodies against blood stages, and/or of vaccines or antibodies against sexual stages. It is, however, also envisaged to genetically engineer mosquitoes to express the antibodies of the present invention.

The term “Plasmodium” is used herein in its conventional meaning known to the skilled person to relate to a genus of obligately parasitic unicellular eukaryotes from the phylum Apicomplexa. Preferably, Plasmodium is a human-parasitic plasmodium, more preferably is a causative agent of malaria. Thus, preferably, Plasmodium is Plasmodium falciparum, Plasmodium vivax, Plasmodium ovale curtisi, or Plasmodium ovale wallikeri, Plasmodium malariae, Plasmodium knowlesi, more preferably is Plasmodium falciparum.

In accordance with the above, the term “Plasmodium infection”, as used herein, relates to an infection of a subject with at least one Plasmodium species or genotype of Plasmodium as specified herein above. Preferably, said infection is infection with P. falciparum. Preferably, said Plasmodium infection causes malaria in said subject. Symptoms and diagnostic measures for the diagnosis of malaria are well-known in the art. Preferably, infection includes re-infection.

The term “subject”, as used herein, relates to a vertebrate animal, preferably a mammal, more preferably a human, a mouse, a rat, a guinea pig, a cat, a dog, a horse, a cattle, a sheep, or a goat, most preferably a human. Preferably, the subject is at risk of becoming infected with a Plasmodium, thus, preferably, the subject is located in a region where Plasmodium infections, in particular malaria, are endemic, or said subject has the intention to spend time in such region.

The present invention also relates to a method of preventing a Plasmodium infection, preferably a Plasmodium falciparum infection, comprising contacting said Plasmodium with an antibody according to the present invention. Preferably, said method is a method of preventing a Plasmodium infection, preferably a Plasmodium falciparum infection, in a subject at risk of becoming infected with Plasmodium, comprising contacting said subject with an antibody according to the present invention.

The method of preventing of the present invention, preferably, may be an in vivo or an in vitro method. Preferably, said method is an in vivo method; thus, preferably, the method is a method performed on a human or animal body. The method may be a method of preventing disease, in particular preventing malaria, as specified elsewhere herein. The method may comprise steps in addition to those explicitly mentioned above. For example, further steps may relate, e.g., to identifying a subject at risk of becoming infected by Plasmodium or providing additional preventive measures to the subject, in particular additional malaria prophylaxis. Moreover, one or more of said steps may be performed by automated equipment. Preferably, the aforesaid method is a method of passive immunization.

The present invention also relates to a method for detecting a Plasmodium circumsporozoite protein in a sample, comprising

a) contacting said sample with an antibody according to any one of claims 1 to 8, and thereby

b) detecting said Plasmodium circumsporozoite protein in said sample.

The method for detecting a Plasmodium circumsporozoite protein of the present invention is an in vitro method and may comprise steps in addition to those explicitly mentioned above. For example, further steps may relate, e.g., to providing a sample for step a), and/or to providing treatment to a subject whose sample was diagnosed to comprise Plasmodium circumsporozoite protein. Moreover, one or more of said steps may be performed by automated equipment. Preferably, the method further comprises further step al) detecting binding of said antibody to said Plasmodium circumsporozoite protein.

The term “Plasmodium circumsporozoite protein” is known to the skilled person to relate to a protein produced by members of the genus Plasmodium, which is involved, among others, in the spread of Plasmodium in the mammalian host. Preferably, the Plasmodium circumsporozoite protein is a Plasmodium falciparum circumsporozoite protein, preferably having the amino acid sequence as shown in Uniprot Acc. No. PF3D7_0304600 (entry of Jun. 5, 2019). Preferably, the Plasmodium circumsporozoite protein is comprised by a Plasmodium cell, preferably a Plasmodium circumsporozoite, more preferably on the cell surface. Thus, preferably, the method for detecting a Plasmodium circumsporozoite protein is a method for detecting a Plasmodium circumsporozoite. More preferably, the method for detecting a Plasmodium circumsporozoite protein is a method providing an indication suitable for detecting exposure to Plasmodium, preferably in the diagnosis of malaria. As will be understood by the skilled person, diagnosis of malaria preferably requires expertise of a medical practitioner and may require additional assessments to be made, e.g. with regards to symptoms of malaria.

The term “sample”, as used herein, refers to a sample from a body fluid, preferably, blood, plasma, serum, saliva or urine, or a sample derived, e.g., by biopsy, from cells, tissues or organs, in particular from potentially Plasmodium-infected tissues. More preferably, the sample is a blood, plasma or serum sample, most preferably, a serum sample. Also preferably, the sample is a sample of a mosquito, more preferably comprising a salivary gland of a mosquito. Biological samples can be derived from a subject by techniques well known in the art. For example, blood samples may be obtained by blood taking, while tissue or organ samples are to be obtained, e.g., by biopsy, and samples from mucosal surfaces may be obtained as swabs or as rinse fluids.

The present invention also relates to a method for detecting an antibody suitable for preventing malaria, comprising

a) providing a candidate antibody suspected to be suitable for preventing malaria;

b) determining an affinity of said candidate antibody to a peptide comprising the amino acid sequence NANP (SEQ ID NO:1);

c) determining an affinity of said candidate antibody to a peptide comprising at least one amino acid sequence selected from NVDP (SEQ ID NO:2), NPDP (SEQ ID NO:3), and KQPA (SEQ ID NO:4); and

d) detecting an antibody suitable for preventing malaria based on the results of steps b) and c).

The method for detecting an antibody of the present invention, preferably, is an in vitro method and may comprise steps in addition to those explicitly mentioned above. For example, further steps may relate, e.g., to providing a sample comprising a candidate antibody.

Preferably, the method for detecting an antibody is a method for identifying an antibody particularly useful for prevention of Plasmodium infection as specified herein above. Thus, in this method, an antibody may be provided from any source deemed appropriate by the skilled person, e.g. preferably from antibody repositories, from newly produced hybridoma clones, purified from or comprised in polyclonal sera, or from affinity screens, e.g. of single-chain antibody libraries. Preferably, in this method, the antibody is provided from a source ensuring that the antibody can be produced in sufficient quantity for the intended use.

Also preferably, the method for detecting an antibody is a method for determining immunization success; thus, preferably, the method is used for determining whether production of antibodies according to the present invention was induced by immunization in a subject, which antibodies are predictive of immunization success. Preferably, in such case, the method is preceded by a step of immunizing a subject against a Plasmodium circumsporozoite protein, preferably by administration of an immunogen comprising at least one of the epitopes NANP, NVDP, NPDP, and KQPA, and preferably a step of providing a sample from said subject. Thus, the method for detecting an antibody may e.g. be used in screening for suitable malaria vaccine.

The term “determining an affinity”, as used herein, relates to establishing whether the antibody has affinity to the epitope of interest, i.e. preferably, whether the antibody binds to said epitope. Thus, determining an affinity may be a qualitative determination, a semi-quantitative determination, or a quantitative determination. Preferably, determining an affinity is a semi-quantitative determination or a quantitative determination, more preferably is a quantitative determination. Methods for establishing antibody binding are known in the art. Preferably, determining an affinity comprises determining a dissociation constant, e.g. by one of the methods shown in the Examples elsewhere herein. It is, however also envisaged that affinity is determined semi-quantitatively, e.g. in comparison to an antibody with a known affinity, such as an antibody of the present invention, in particular mAb 4493 and/or mAb 2541. Determining an affinity preferably is accomplished in a method directly determining interaction of the antibody with a peptide comprising the epitope of interest, e.g. in an ELISA. Determining an affinity may, however, also be accomplished in a method indirectly determining interaction of the antibody with a peptide comprising the epitope of interest, e.g. in a competitive assay. As the skilled person will understand, the latter method may be preferable in particular in a case where the antibody of interest is comprised in a preparation comprising further antibodies, e.g. a serum sample. Thus, in such case, preferably, the method for determining whether antibodies according to the present invention were induced by immunization in a subject, step a) may comprise providing a serum sample, step b) may comprise determining interaction of the antibodies in the sample with a peptide comprising the amino acid sequence NANP, and step c) may comprise determining interaction of the antibodies in the sample with a peptide comprising the amino acid sequence NANP in the presence of a peptide comprising at least one amino acid sequence selected from NVDP, NPDP, and KQPA; as the skilled person understands, step b) may also comprise determining interaction of the antibodies in the sample with a peptide comprising at least one amino acid sequence selected from NVDP, NPDP, and KQPA in the presence of a peptide comprising the amino acid sequence NANP, and step c) may comprise determining interaction of the antibodies in the sample with a peptide comprising at least one amino acid sequence selected from NVDP, NPDP, and KQPA. Also in particular in a case where the antibody of interest in comprised in a preparation comprising further antibodies, e.g. a serum sample, step b) or c) may comprise a step of affinity purifying antibodies having affinity for a peptide comprising the amino acid sequence NANP, or for a peptide comprising at least one amino acid sequence selected from NVDP, NPDP, and KQPA, respectively, and using said affinity-purified antibodies in step c) or b) as appropriate. As will be clear from the above, in particular in case the method is a method for determining immunization success, it is preferably sufficient for identifying an antibody suitable for preventing malaria to establish that such an antibody is present in the sample, while its isolation preferably is not required. Preferably, determining the affinity of a candidate antibody comprises determining relative or absolute, preferably absolute, affinity; more preferably, determining the affinity of a candidate antibody comprises determining the value of a dissociation constant K_{D} for the candidate antibody and the peptide. Preferably, an antibody suitable for preventing malaria is identified if (i) it is determined in step b) that the candidate antibody has high affinity to a peptide comprising the amino acid sequence NANP, preferably an affinity a specified elsewhere herein; and (ii) it is determined in step c) that the candidate antibody has high affinity to a peptide comprising at least one amino acid sequence selected from NVDP, NPDP, and KQPA, preferably an affinity a specified elsewhere herein. Thus, more preferably, an antibody suitable for preventing malaria is identified if (i) it is determined in step b) that the dissociation constant K_{D} for the candidate antibody and the peptide comprising an amino acid sequence NANP is at most 10⁻⁶ M, preferably at most 2×10⁻⁷ M, more preferably at most 10⁻⁷ M, even more preferably at most 5×10⁻⁸ M, still more preferably at most 2×10⁻⁸ M, most preferably at most 10⁻⁸ M; and (ii) it is determined in step c) that the dissociation constant K_{D} for the candidate antibody and the peptide comprising an amino acid sequence NVDP, NPDP, and KQPA is at most 10⁻⁶ M, preferably at most 2×10⁻⁷ M, more preferably at most 10⁻⁷ M, even more preferably at most 5×10⁻⁸ M, still more preferably at most 2×10⁻⁸ M, most preferably at most 10⁻⁸ M and/or the dissociation constant K_{D} for the candidate antibody and the peptide comprising an amino acid sequence KQPA is at most 10⁻⁵ M, preferably at most 5×10⁻⁶ M, more preferably at most 2×10⁻⁶ M, most preferably at most 10⁻⁷ M. It is, however, also envisaged that the affinity of the candidate antibody is determined by determining similarity to known antibodies of the present invention, in particular mAb 4493 or mAb 2541, by determining the sequence at least of one, more preferably at least two, more preferably at least three, most preferably all six, CDRs, of said candidate antibody, e.g. by protein sequencing, by sequencing of the encoding nucleic acid sequence(s), or via mass spectrometry.

The present invention also relates to a method for improving an anti-Plasmodium antibody, preferably an anti-Plasmodium circumsporozoite antibody, comprising

A) providing at least one derivative antibody, preferably a multitude of derivative antibodies, of an antibody binding to a peptide comprising the amino acid sequence NANP (SEQ ID NO:1);

B) determining the affinity of said derivative antibody to a peptide comprising at least one amino acid sequence selected from NVDP (SEQ ID NO:2), NPDP (SEQ ID NO:3), and KQPA (SEQ ID NO:4);

C) selecting as an improved anti-Plasmodium antibody a derivative antibody having an increased affinity to at least one of amino acid sequences NVDP, NPDP, and KQPA compared to said antibody binding to a peptide comprising the amino acid sequence NANP.

The method for improving an anti-Plasmodium antibody is an in vitro method and may comprise steps in addition to those referred to above. Moreover, one or more of said steps may be performed by automated equipment.

The term “improving an anti-Plasmodium antibody” as used herein, relates to an improvement of said antibody with regards to its suitability in the prevention of a Plasmodium infection as specified elsewhere herein. Thus, improving preferably is increasing the affinity of said antibody to a peptide comprising at least one amino acid sequence selected from NVDP, NPDP, and KQPA.

The term “providing a derivative antibody” relates to providing an antibody non-identical to, but based on, an antibody binding to a peptide comprising the amino acid sequence NANP. Preferably, said derivative antibody is provided by chemical derivatization, preferably by glycosylation or by a similar addition of one or more chemical groups to the antibody. Chemical derivatization may, however, also comprise removal of chemical groups from the antibody, e.g. deglycosylation or removal of the Fc portion of an antibody. More preferably, a derivative is provided by exchanging, adding and/or deleting, preferably exchanging, at least one amino acid in the amino acid sequence of the antibody for a non-identical amino acid. The exchange may be introduced into any polypeptide constituting the antibody, in particular in a heavy chain, in a light chain, or in a polypeptide constituting a single-chain antibody. Preferably, at least one amino acid in at least one CDR sequence is exchanged, added, and/or deleted, preferably exchanged. More preferably, providing a derivative antibody comprises increasing the number of aromatic amino acids in the CDRs, preferably at (a) position(s) corresponding to position 32 and/or 96 of SEQ ID NO:30 and/or at (a) position(s) corresponding to position 50 and/or 100 in SEQ ID NO:29; or at (a) position(s) corresponding to position 94 and/or 96 in SEQ ID NO: 32 and/or at (a) position(s) corresponding to position 52, 58, 97, 98, and/or 100 in SEQ ID NO:31. Methods for providing the aforesaid derivative antibodies are well known in the art and include preferably random mutagenesis, site directed mutagenesis, as well as model-directed mutagenesis, in particular based on the structural data provided in the Examples herein, of one or more polynucleotides encoding polypeptides constituting the antibody. As the skilled person understands, antibody libraries, e.g. single-chain antibody libraries, may also be used for providing derivatives of an antibody. Preferably, the derivative antibody is an antibody variant as specified herein above.

The present invention also relates to a kit comprising (i) an antibody according to the present invention and/or a polynucleotide or polynucleotides encoding an antibody according to the present invention; comprised in a housing; to a kit for diagnosing malaria comprising the antibody according to according to the present invention and an agent for detection of binding of said antibody to its epitope on a Plasmodium circumsporozoite protein, preferably on a Plasmodium cell; and to a kit for determining the quality of an immune response of a subject to a Plasmodium circumsporozoite protein comprising a peptide comprising an amino acid sequence NANP (SEQ ID NO:1) and at least one peptide comprising an amino acid sequence selected from NVDP (SEQ ID NO:2), NPDP (SEQ ID NO:3), and KQPA (SEQ ID NO:4).

The term “kit”, as used herein, refers to a collection of the aforementioned means and optionally instructions, provided preferably in a ready-to-use manner. The means are, preferably, provided in a single container (i.e. a housing). Preferably, the kit is for use according to a method of the present invention; thus, preferably, the kit also comprises further components, which are necessary for carrying out the method. Such components preferably are auxiliary agents, which are required for the detection of antibody binding, agents for pre-treating the sample to be analyzed, calibration standards, or negative and/or positive controls such as the antibodies of the present invention.

Further, the present invention relates to a device for detecting Plasmodium infection in a sample comprising:

a) an analyzing unit comprising the antibody according to the present invention; and

b) a detector which detects binding of the antibody in the analyzing unit to its epitope on a Plasmodium circumsporozoite protein, preferably on a Plasmodium cell.

The term “device”, as used herein, relates to a system comprising at least the aforementioned analyzing unit and detector unit, which detects binding of the antibody, operatively linked to each other. Preferably, the device further comprises an evaluation unit evaluating the results of detection step b). How to link the units of the device in an operating manner will depend on the type of units included into the device. For example, where units for automatic analysis of a sample are applied, the data obtained by said automatically operating analyzing unit and/or detector can be processed by, e.g., a computer program in order to obtain the desired results by the evaluation unit. Preferably, the units are comprised by a single device in such a case. The analyzing unit may comprise the antibody in immobilized form on a solid support. Such an analyzing unit is particular useful for liquid samples. The sample to be investigated with the device of the present invention is preferably a tissue sample or a blood, plasma, or serum sample. In another aspect, the antibody may be comprised in a detection solution, which will be applied to tissue samples such as tissue section by the analyzing unit. The detection solution can be stored in the analyzing unit or a separate vial, even outside the device. The evaluation unit, preferably a computer or data processing device, comprises implemented rules, i.e. an algorithm, for evaluating the binding determined by the analyzing unit whereby the binding preferably is evaluated into significant or non-significant binding based on the signal type, strength and, in the case of tissue samples, position of the signal with respect to the tissue.

The present invention also relates to a use of an antibody according to the present invention or a polynucleotide or polynucleotides according to the present invention for inhibiting Plasmodium infection, preferably in vitro inhibiting Plasmodium infection.

In view of the above, the following embodiments are particularly envisaged:

1. An antibody binding to a peptide comprising an amino acid sequence NANP (SEQ ID NO:1) and to at least one peptide comprising an amino acid sequence selected from NVDP (SEQ ID NO:2), NPDP (SEQ ID NO:3), and KQPA (SEQ ID NO:4), in a preferred embodiment wherein the peptide comprising the amino acid sequence NANP consists of the amino acid sequence NPNANPNANPNANPNANPNANP (SEQ ID NO:44) or NANPNANPNANPNANPNANP (SEQ ID NO:45), wherein the peptide comprising the amino acid sequence KQPA consists of the amino acid sequence KQPADGNPDPNANPN (SEQ ID NO:37), wherein the peptide comprising the amino acid sequence NPDP consists of the amino acid sequence NPDPNANPNVDPNANP (SEQ ID NO:38), and wherein the peptide comprising the amino acid sequence NVDP consists of the amino acid sequence NVDPNANPNVDPNANPNVDP (SEQ ID NO:39).

2. The antibody of embodiment 1, wherein the dissociation constant K_{D} for the antibody and the peptide comprising an amino acid sequence NANP is at most 10⁻⁶M, preferably at most 2×10⁻⁷ M, more preferably at most 10⁻⁷ M, even more preferably at most 5×10⁻⁸M, still more preferably at most 2×10⁻⁸ M, most preferably at most 10⁻⁸ M.

3. The antibody of embodiment 1 or 2, wherein the dissociation constant K_{D} for the antibody and the peptide comprising an amino acid sequence NVDP or NPDP is at most 10⁻⁶ M, preferably at most 2×10⁻⁷M, more preferably at most 10⁻⁷M, even more preferably at most 5×10⁻⁸ M, still more preferably at most 2×10⁻⁸ M, most preferably at most 10⁻⁸ M.

4. The antibody of any one of embodiments 1 to 3, wherein the dissociation constant K_{D} for the antibody and the peptide comprising an amino acid sequence KQPA is at most 10⁻⁵ M, preferably at most 5×10⁻⁶ M, more preferably at most 2×10⁻⁶ M, most preferably at most 10⁻⁷ M.

5. The antibody of any one of embodiments 1 to 4, wherein said antibody comprises complementarity determining regions (CDRs) comprising the sequences of SEQ ID Nos: 5 to 10 or SEQ ID Nos:11 to 16, or, in a preferred embodiment, comprises complementarity determining regions (CDRs) comprising sequences at least 80% identical to the sequences of SEQ ID NOs:5 to 10 or SEQ ID NOs:11 to 16.

6. The antibody of any one of embodiments 1 to 5, wherein said antibody comprises
(i) an amino acid sequence of the heavy chain as shown in SEQ ID NO:29 or a sequence at least 50% identical to SEQ ID NO:29; and an amino acid sequence of the light chain as shown in SEQ ID NO:30 or a sequence at least 50% identical to SEQ ID NO:30; or
(ii) an amino acid sequence of the heavy chain as shown in SEQ ID NO:31 or a sequence at least 50% identical to SEQ ID NO:31; and an amino acid sequence of the light chain as shown in SEQ ID NO:32 or a sequence at least 50% identical to SEQ ID NO:33.

7. The antibody of any one of embodiments 1 to 6, wherein said antibody is a monclonal antibody or a fragment thereof.

8. The antibody of any one of embodiments 1 to 7, wherein said antibody is a human or a humanized antibody, preferably is a human antibody.

9. An antibody according to any one of embodiments 1 to 8 for use in medicine.

10. An antibody according to any one of embodiments 1 to 8 for use in prevention of Plasmodium infection, preferably a Plasmodium falciparum infection, preferably for use in prevention of malaria.

11. A method of preventing a Plasmodium infection, preferably a Plasmodium falciparum infection, preferably in a subject at risk of becoming infected with a Plasmodium, comprising contacting said Plasmodium with an antibody according to any one of embodiments 1 to 8.

12. The method of embodiment 11, wherein said Plasmodium infection causes malaria in said subject.

13. The method of embodiment 11 or 12, wherein said method is a method of preventing malaria.

14. A method for detecting a Plasmodium circumsporozoite protein in a sample, comprising
a) contacting said sample with an antibody according to any one of embodiments 1 to 8, and thereby
b) detecting said Plasmodium circumsporozoite protein in said sample.

15. The method of embodiment 14, wherein said method comprises further step al) detecting binding of said antibody to said Plasmodium circumsporozoite protein.

16. The method of embodiment 14 or 15, wherein said Plasmodium circumsporozoite protein is comprised in a Plasmodium cell, preferably a Plasmodium circumsporozoite.

17. The method of anyone of embodiments 14 to 16, wherein said Plasmodium is Plasmodium falciparum.

18. A method for detecting an antibody suitable for preventing malaria, comprising
a) providing a candidate antibody suspected to be suitable for preventing malaria;
b) determining an affinity of said candidate antibody to a peptide comprising the amino acid sequence NANP (SEQ ID NO:1);
c) determining an affinity of said candidate antibody to a peptide comprising at least one amino acid sequence selected from NVDP (SEQ ID NO:2), NPDP (SEQ ID NO:3), and KQPA (SEQ ID NO:4); and
d) identifying an antibody suitable for preventing malaria based on the results of steps b) and c).

19. The method of embodiment 18, wherein determining the affinity of a candidate antibody comprises semiquantitative or quantitative, preferably quantitative, determination of said affinity.

20. The method of embodiment 18 or 19, wherein determining the affinity of a candidate antibody comprises determining relative or absolute, preferably absolute, affinity.

21. The method of any one of embodiments 18 to 20, wherein determining the affinity of a candidate antibody comprises determining the value of a dissociation constant K_{D} for the candidate antibody and the peptide.

22. The method of any one of embodiments 18 to 21, wherein an antibody suitable for preventing malaria is identified if (i) it is determined in step b) that the candidate antibody has high affinity to a peptide comprising the amino acid sequence NANP; and (ii) it is determined in step c) that the candidate antibody has high affinity to a peptide comprising at least one amino acid sequence selected from NVDP, NPDP, and KQPA.

23. The method of any one of embodiments 18 to 22, wherein an antibody suitable for preventing malaria is identified if (i) it is determined in step b) that the dissociation constant K_{D} for the candidate antibody and the peptide comprising an amino acid sequence NANP is at most 10⁻⁶ M, preferably at most 2×10⁻⁷ M, more preferably at most 10⁻⁷ M, even more preferably at most 5×10⁻⁸ M, still more preferably at most 2×10⁻⁸ M, most preferably at most 10⁻⁸ M; and (ii) it is determined in step c) that the dissociation constant K_{D} for the candidate antibody and the peptide comprising an amino acid sequence NVDP, NPDP, and KQPA is at most 10⁻⁶ M, preferably at most 2×10⁻⁷ M, more preferably at most 10⁻⁷ M, even more preferably at most 5×10⁻⁸ M, still more preferably at most 2×10⁻⁸ M, most preferably at most 10⁻⁸ M and/or the dissociation constant K_{D} for the candidate antibody and the peptide comprising an amino acid sequence KQPA is at most 10⁻⁵ M, preferably at most 5×10⁻⁶ M, more preferably at most 2×10⁻⁶ M, most preferably at most 10⁻⁷ M.

24. A method for improving an anti-Plasmodium antibody, preferably an anti-Plasmodium circumsporozoite antibody, comprising
A) providing at least one derivative antibody, preferably a multitude of derivative antibodies, of an antibody binding to a peptide comprising the amino acid sequence NANP (SEQ ID NO:1);
B) determining the affinity of said derivative antibody to a peptide comprising at least one amino acid sequence selected from NVDP (SEQ ID NO:2), NPDP (SEQ ID NO:3), and KQPA (SEQ ID NO:4);
C) selecting as an improved anti-Plasmodium antibody a derivative antibody having an increased affinity to at least one of amino acid sequences NVDP, NPDP, and KQPA compared to said antibody binding to a peptide comprising the amino acid sequence NANP.

25. The method of embodiment 24, wherein said derivative antibody comprises at least one amino acid exchange in at least one complementarity determining region (CDR) of the antibody binding to a peptide comprising the amino acid sequence NANP.

26. A kit comprising (i) an antibody according to any one of embodiments 1 to 8 and/or a polynucleotide or polynucleotides encoding an antibody according to any one of embodiments 1 to 8; comprised in a housing.

27. A device for diagnosing malaria in a sample comprising:

a) an analyzing unit comprising the antibody according to any one of embodiments 1 to 8; and

b) a detector which detects binding of the antibody in the analyzing unit to its epitope on a Plasmodium circumsporozoite protein, preferably on a Plasmodium cell.

28. A kit for diagnosing malaria comprising the antibody according to any one of embodiments 1 to 8 and an agent for detection of binding of said antibody to its epitope on a Plasmodium circumsporozoite protein, preferably on a Plasmodium cell.

29. A kit for determining the quality of an immune response of a subject to a Plasmodium circumsporozoite protein comprising a peptide comprising an amino acid sequence NANP (SEQ ID NO:1) and at least one peptide comprising an amino acid sequence selected from NVDP (SEQ ID NO:2), NPDP (SEQ ID NO:3), and KQPA (SEQ ID NO:4).

30. The kit of embodiment 29, wherein said kit further comprises an agent for detecting an antibody.

31. A polynucleotide or polynucleotides encoding the antibody according to any one of embodiments 1 to 8.

32. Use of an antibody according to any one of embodiments 1 to 8 or a polynucleotide or polynucleotides according to embodiment 31 for inhibiting Plasmodium infection, preferably in vitro inhibiting Plasmodium infection.

All references cited in this specification are herewith incorporated by reference with respect to their entire disclosure content and the disclosure content specifically mentioned in this specification.

### FIGURE LEGENDS

FIG. 1. Cross-reactivity of human PfCSP-reactive antibodies

(A) Schematic representation of PfCSP from NF54 comprising the N-terminus, central repeat, and C-terminal (C-CSP) domain. The amino acid sequence downstream of the N-terminal domain including the conserved region 1 (RI), the N-terminal junction, and the central repeat domain is indicated, as well as the NANA epitope in the linker region upstream of the aTSR domain in C-CSP. Amino acid sequences of overlapping peptides in the N-terminal junction containing known epitopes of protective antibodies (Kisalu et al., 2018, Tan et al., 2018), designated as KQPA, NPDP, NVDP, and of a NANP 5.5-mer peptide (designated as NANP) are indicated and shown in different shades of grey.

(B) Binding strength of anti-PfCSP antibodies (n=200; Murugan et al., 2018) to the indicated overlapping peptides and C-CSP as in (A) is shown as calculated area under curve (AUC) values based on ELISA measurements at different antibody concentrations. The frequency of reactive and non-reactive antibodies is indicated.

(C and D) Binding profile to the indicated PfCSP peptides and C-CSP as in (B) of representative antibodies with specificity for NVDP, NANP, or C-CSP (C) and cross-reactive antibodies with different binding profiles (D).

Data in B-D shows mean values from three independent experiments. Horizontal lines in B-D indicate the reactivity threshold.

FIG. 2. Cross-reactivity of NANP antibodies with the N-terminal junction but not C-CSP is associated with high affinity

(A-C) Affinity of epitope-specific compared to cross-reactive antibodies as determined by SPR.

(A) NVDP affinity (left) of NVDP-specific antibodies (n=16) and of NVDP, NPDP cross-reactive antibodies (n=22) and NPDP affinity of NVDP, NPDP cross-binders (right).

(B) NANP (left), NVDP (center left), NPDP (center right) and KQPA (right) affinities of NANP-specific antibodies (n=41) and of NANP binders with cross-reactivity to NVDP (n=24), NVDP and NPDP (n=19), or NVDP, NPDP and KQPA (n=6).

(C) C-CSP (left), NANP (center left), NVDP (center right) and NPDP (right) affinities of C-CSP-specific antibodies (n=4) and C-CSP binders with cross-reactivity to NANP (n=9), NANP and NVDP (n=5), or NANP, NVDP and NPDP (n=3).

(A-C) Black horizontal lines indicate geometric means. P-values were calculated by Mann-Whitney test. *P<0.05; **P<0.01; ***P<0.001; ****P<0.0001; ns indicates statistically non-significant differences.

FIG. 3: The paratope core of cross-reactive PfCSP antibody 4493 preferentially binds NANP motifs.

(A) Affinity of clonally related antibodies (4142, 4493 and 4560) with different numbers of

IGH and IGL somatic mutations (aa exchanges) to the indicated peptides determined by Surface Plasmon Resonance (SPR).

(B) Affinity of mAb 4493 to the indicated peptides as measured by isothermal titration calorimetry (ITC).

(C-G) mAb 4493 in co-complex with the peptides KQPA (C), NPDP (D), NDN₃ (E), DND₃

(F) and NANP (G) H-bonds between mAb 4493 and the respective peptide are shown.

(H) Positioning of the 4-aa motifs in the indicated peptides in the mAb 4493 paratope as observed in the antibody co-complexes (C-G). Three distinct paratope positions (0, 1 and 2) are indicated. Amino acid residues resolved in the X-ray crystal structures are underlined.

FIG. 4: High-affinity cross-reactive antibodies are potent parasite inhibitors

(A-C) Capacity of antibodies (n=139, 100 μg/ml) with the indicated binding profiles to inhibit the hepatocyte traversal activity of Pf sporozoites in vitro.

(A) NVDP-specific antibodies (n=14) and NVDP, NPDP cross-reactive antibodies (n=18).

(B) NANP-specific antibodies (n=41) and NANP binders with cross-reactivity to NVDP (n=20), NVDP and NPDP (n=18), or NVDP, NPDP and KQPA (n=6).

(C) C-CSP-specific antibodies (n=5) and C-CSP binders with cross-reactivity to NANP (n=9), NANP and NVDP (n=5), or NANP, NVDP and NPDP (n=3).

(D) IC₅₀ values versus NANP (left), NVDP (middle), and NPDP (right) of selected antibodies with >95% Pf hepatocyte traversal-inhibitory activity (B and C).

(A-C) Black horizontal lines indicate arithmetic mean. (A-C) Data is representative of at least two independent experiments. (D) IC₅₀ values were calculated from data of at least three independent experiments. P-values were calculated by Mann-Whitney test. *P<0.05; **P<0.01; ***P<0.001; ****P<0.0001; ns indicates non-significant statistical differences.

FIG. 5. In vivo protective activity of cross-reactive IGHV3-33- and non-IGHV3-33-encoded PfCSP antibodies with different binding profiles

(A) Scheme and time line of the experiments. Antibody potency was assessed in C57BL/6 mice after intra peritoneal (i.p.) passive transfer of mAb 20 h before exposure to bites of mosquitoes infected with Plasmodium berghei transgenic parasites expressing P. falciparum CSP (PbPfCSP). Two to three hours post infection (hpi) blood was collected and serum concentrations of the monoclonal antibodies were determined by ELISA. The time to blood-stage parasitemia was monitored by blood smear between day 3 and day 10 after the mosquito-bite exposure.

(B-D) In vivo protective activity of cross-reactive IGHV3-33-encoded PfCSP antibodies with different binding profiles.

(B) Affinity profiles of mAbs 1210 (Imkeller et al., 2018), 2164 and 4476 to KQPA, NPDP, NVDP, NANP and C-CSP as determined by SPR.

(C) Capacity of the indicated antibodies (300 μg/ml) to protect mice from infection by mosquito bites with PbPfCSP parasites in three independent experiments as determined by the percentage of parasite-free mice. The total number of mice per antibody in three experiments is indicated (n).

(D) Serum concentration of the transferred monoclonal antibodies in individual mice at the time of parasite challenge.

(E-G) In vivo protective activity of cross-reactive non-IGHV3-33-encoded PfCSP antibodies with different binding profiles.

(E) Affinity profiles of mAbs 4493, CIS43 (Kisalu et al., 2018), and 317 (Oyen et al., 2017) to KQPA, NPDP, NVDP, NANP and C-CSP as determined by SPR in comparison to the IGHV3-33-encoded mAb 1210 (Imkeller et al., 2018).

(F) Capacity of the indicated antibodies (150 μg/ml) to protect mice from infection by mosquito bites with PbPfCSP parasites in two independent experiments as determined by the percentage of parasite-free mice. The total number of mice per antibody in two experiments is indicated (n).

(G) Serum concentrations of the transferred monoclonal antibodies in individual mice at the time of parasite challenge.

(H) Affinity profile of mAb 2541 (filled circle) to the indicated peptides compared to the other IGHV3-33-, IGKV1-5-encoded plasmablast antibodies (open circles) as determined by SPR.

(I) All mAbs as in (F) and mAb 2541 were tested for their capacity to protect from blood-stage parasitemea after passive i.p. mAb transfer of 150 μg/mouse. Pooled data from three independent experiments is shown. The total number of mice per group is indicated (n). (J) Serum concentration of the transferred monoclonal antibodies in individual mice at the time of parasite challenge.

(B, E) Data represent the mean from three independent measurements. (D, G, J) Data represent the mean from at least two independent measurements. (C, F, I) mAbs showing statistically significant difference (P<0.05) in protection are indicated with different alphabets (a, b, c). (D, G, J) Horizontal black lines indicate means. P-values were calculated by Mantel-Cox log-rank test (C, F, I) and Mann-Whitney test (G). ***P<0.001.

The following Examples shall merely illustrate the invention. They shall not be construed, whatsoever, to limit the scope of the invention.

### EXAMPLE 1: MATERIALS

### Cell Lines

HEK293F cells were cultured according to the manufacturer's instructions. The cells were passaged at 37° C., 8% CO₂ and 180 rpm in a 50 ml Bioreactor in FreeStyle™ 293-F medium. HC-04 cells (MRA-975, deposited by Jetsumon Sattabongkot; Sattabongkot et al., 2006) were cultured at 37° C. and 5% CO₂ using HC-04 complete culture medium (428.75 ml MEM (-L-glu), 428.75 ml F-12 Nutrient Mix (+L-glu), 15 mM HEPES, 1.5 g/l NaHCO₃, 2.5 mM L-glutamine and 10% FCS).

### Bacteria

MAX Efficiency® DH10B™ Competent Cells were cultured at 37° C. and 180 rpm in LB medium for maintenance and Terrific broth for plasmid production.

Plasmodium falciparum Cultures

Plasmodium falciparum PfNF54 (a kind gift of Prof. R. Sauerwein) were cultured in O+ human red blood cells at 37° C., 4% CO₂ and 3% 02 in a Heracell 150i Tri-gas incubator (Thermo Scientific). For gametocyte production, asynchronous parasite cultures were diluted to 1% parasitaemia and maintained for 15-16 days with daily change of RPMI-1640 medium (Thermo Scientific cat #52400) supplemented with 10% human A+ serum and 10 mM hypoxantine (c-c-Pro) until mosquito infections.

Pb-PfCSP, a replacement P. berghei line expressing Pf CSP (NF54) under the control of the Pb csp regulatory sequences (Triller et al. 2017), was obtained from Chris J. Janse and Shahid M. Khan and passaged every 3-4 days in CD1 female mice.

### Mosquitoes

All mosquitoes were kept at 28-30° C. and 70-80% humidity. Anopheles coluzzii Ngousso S1 strain (Harris et al, 2010) were used for the production of Pf NF54 sporozoites for in vitro traversal assays. A. gambiae 7b line, an immunocompromised transgenic mosquitoes derived from the G3 laboratory strain (Pompon and Levashina, 2015), were used for the production of Pb-PfCSP sporozoites for in vivo infections.

### Mice

Female C57BL/6 mice (7-9 weeks old) and female CD-1 mice (8-12 weeks old) were bred in the MPIIB Experimental Animal Facility (Marienfelde, Berlin), handled in accordance with the German Animal Protection Law (§ 8 Tierschutzgesetz) and approved by the Landesamt für Gesundheit and Soziales (LAGeSo), Berlin, Germany (project numbers 368/12 and H0335/17).

### EXAMPLE 2: METHODS

### Ig Gene Cloning and Recombinant Antibody Production

Ig heavy and light chain genes corresponding to antibody were cloned into human Igγ1 (AbVec2.0-IGHG1, Genbank ID: LT615368.1) and Igκ (AbVec1.1-IGKC, Genbank ID: LT615369.1) or Igλ (AbVec1.1-IGLC2-XhoI) expression vectors, respectively (Tiller et al., 2009). The cloning vectors are available from Addgene (Catalog numbers: 80795, 80796 and 99575). In brief, restriction site-tagged specific V and J-gene primers were used for amplifying Ig genes from single B cells and the amplicons were cloned into the above mentioned vectors. Ig genes of mAbs CIS43 and 317 were obtained by reverse translation of the protein sequences deposited (PDB accession number 6B5M for CIS43 (Kisalu et al., 2018) and 6AXL for 317 (Oyen et al., 2017)). Ig genes of CIS43 were synthesized at MWG Eurofins Genomics with AgeI restriction site at the 5′ end and SalI and BsiWI restriction sites at the 3′end of the heavy and kappa Ig genes, respectively. Ig genes of 317 were synthesized at GeneArt (Thermofisher) and restriction sites were introduced via PCR. Upon successful cloning, recombinant monoclonal antibodies were expressed in HEK293F cells (ThermoFisher Scientific).

### Enzyme-Linked Immunosorbent Assay

Recombinant monoclonal antibodies were purified using Protein G Sepharose beads (GE healthcare) and the IgG concentration was measured by ELISA as described (Tiller et al., 2008). Antigen and serum ELISAs were performed as described (Triller et al., 2017). In brief, high-binding 384 well polystyrene plates (Corning) were coated overnight at 4° C. with KQPA, NPDP, NVDP, C-CSP or Streptavidin at 50 ng/well or PfCSP at 40 ng/well in 25 μl. Streptavidin-coated plates were incubated for 1 h with 200 ng/well biotinylated NANP5.5 in 25 μl. Plates were washed 3 times with 0.05% Tween 20 in PBS, blocked with 50 μl of 1% BSA in PBS for 1 h at room temperature (RT), and washed again prior to incubation with monoclonal antibodies at the indicated concentrations for 1.5 h at RT. Wells were washed and incubated with goat anti-human IgG-HRP (Jackson Immuno Research) in PBS with 1% BSA. One-step ABTS substrate (RT, 20 μl/well; Roche) and 1×KPL ABTS® peroxidase stop solution (RT, 20 μl/well; SeraCare Life Sciences, Inc.) were used for detection. A chimeric version of the murine anti-PfCSP antibody 2A10 (Triller et al., 2017) with human Ig heavy and Ig kappa constant regions and the non-PfCSP-reactive antibody mGO53 (Wardemann et al., 2003) were used as a positive control and negative controls, respectively. ELISA area under curve (AUC) values were calculated using GraphPad Prism 7.04 (GraphPad).

### Surface Plasmon Resonance (SPR)

SPR measurements were performed with a BIACORE T200 (GE Healthcare) as described (Murugan et al., 2018). In brief, the instrument was docked with a series S sensor chip CMS (GE Healthcare). 10 mM HEPES with 150 mM NaCl at pH 7.4 was used as running buffer. All samples were immobilized by amine coupling using the human antibody capture kit (GE Healthcare) according to the manufacturer's instructions. Sample antibodies and the non-PfCSP-reactive negative control antibody mGO53 (Wardemann et al., 2003) were captured in the sample and reference flow cell at equal concentrations, respectively. Flow cells were stabilized with running buffer at 10 μl/min flowrate for 20 min. The respective peptides were dissolved in running buffer and injected at 0, 0.015, 0.09, 0.55, 3.3, and 20 μM concentration. A flow rate of 30 μl/min was maintained, allowing the association and dissociation of the peptides for 60 s and 180 s respectively, at 25° C. For high affinity antibodies (˜10⁻¹⁰ M), additional measurement at 0, 0.42, 2.57, 15.43, 92.6 and 555.5 nM concentration was performed. After each run, both flow cells were regenerated with 3 M MgCl₂. The data were fit using 1:1 binding model or steady state kinetic analysis using the BIACORE T200 software V2.0.

### Pf Sporozoite Hepatocyte Traversal Assay

Anopheles coluzzii mosquitoes were infected with mature Pf gametocytes (NF54 strain) via artificial midi-feeders (Glass Instruments, The Netherlands) for 15 min and kept at 26° C. and 80% humidity in a controlled S3 facility in accordance with local safety authorizations (Landesamt für Gesundheit and Soziales Berlin, Germany, LAGeSo, project number 411/08). Infected mosquitoes received an additional uninfected blood meal 7-8 days post infection (dpi) and were collected 13-15 dpi to isolate sporozoites. Sporozoites were isolated in HC-04 medium by dissecting and grinding mosquito thoraces containing salivary glands with glass pestles, followed by filtering the extracts with 100 μm and 40 μm cell strainers. The isolated salivary gland sporozoites were enumerated in a hemocytometer (Malassez, Marienfelde) and used for traversal assays as previously described (Triller et al., 2017). Briefly, salivary gland Pf sporozoites in HC-04 medium were pre-incubated with 100 μg/ml or serial dilutions (0.032, 0.16, 0.8, 4 and 20 μg/ml) of monoclonal antibodies in 27.5 μl for 30 min on ice and added to human hepatocytes (HC-04, (Sattabongkot et al., 2006)) for 2 h at 37° C. and 5% CO₂ in the presence of 0.5 mg/ml dextran-rhodamine (Molecular Probes). Cells were washed and fixed with 1% PFA in PBS before measuring dextran positivity using FACS LSR II instrument (BD Biosciences). Data analysis was performed by subtraction of the background (dextran positive cells after incubation with uninfected mosquito salivary gland material) and normalization to the maximum Pf traversal capacity (dextran positive cells after incubation with salivary gland Pf sporozoites) using FlowJo V.10.0.8 (Tree Star). A chimeric humanized version of the PfCSP-reactive monoclonal antibody 2A10 (Triller et al., 2017) and of the non-PfCSP-reactive monoclonal antibody mGO53 (Wardemann et al., 2003) was used as positive and negative control, respectively. IC₅₀ values were calculated for each antibody by four-parameter logistic curve fitting in GraphPad Prism 7.04 (GraphPad) using the measurements from at least three independent experiments.

### Fab Production

Fabs of mAbs 2243, 4498, 2164, 4476 and 3945 were generated by papain digestion of IgG, purified via Protein A chromatography followed by cation-exchange chromatography (MonoS, GE Healthcare) and size-exclusion chromatography (Superdex 200 Increase 10/300 GL, GE Healthcare). Fabs of mAb 4493 were generated by cloning of the IGH and IGK variable region gene segments into pcDNA3.4 TOPO expression vectors immediately upstream of human IGK and CHI constant regions, respectively, followed by transient expression in HEK293F cells (Thermo Fisher Scientific) and purification via KappaSelect affinity chromatography (GE Healthcare), cation-exchange chromatography (MonoS, GE Healthcare) and size-exclusion chromatography (Superdex 200 Increase 10/300 GL, GE Healthcare).

### Crystallization and Structure Determination

To enhance crystallizability, purified 4493 Fabs were mixed with the anti-kappa V_{H}H domain (Thermofisher) in a 1:2 molar ratio and excess V_{H}H domain was removed away via size exclusion chromatography (Superdex 200 Increase 10/300 GL, GE Healthcare). Fab-V_{H}H co-complexes were concentrated to 6 mg/mL and diluted to 5 mg/mL with the respective peptide. 4493-V_{H}H-KQPA crystals grew in 0.1 M HEPES pH 7.0, 1 M lithium chloride, 20% (w/v) PEG 6000 and were cryoprotected in 15% (w/v) ethylene glycol. 4493-V_{H}H-NPDP crystals grew in 20% (w/v) PEG 3350, 0.2 M sodium nitrate and were cryoprotected in 15% (w/v) ethylene glycol. 4493-V_{H}H-NDN₃ crystals grew in 20% (w/v) PEG 3350, 0.2 M potassium nitrate and were cryoprotected in 15% (w/v) ethylene glycol. 4493-V_{H}H-DND₃ crystals grew in 20% (w/v) PEG 8000, 0.1 M IVIES pH 6.0 and 0.2 M calcium acetate and were cryoprotected in 20% (w/v) glycerol. 4493-V_{H}H-NANP3 grew in 40% (w/v) PEG 600, 0.1 M sodium citrate pH 5.5 after microseeding from thin needle-like crystals that grew in 0.1 M HEPES pH 7.5, 20% (w/v) PEG 8000 and were cryoprotected in 15% (w/v) ethylene glycol. Data were collected at the 08ID-1 beamline at the Canadian Light Source (CLS), the 23-ID beamline at the Advanced Photon Source (APS), or the 17-ID-2 beamline at the National Synchrotron Light Source II (NSLS-II) processed and scaled using XDS (Kabsch et al., 2010). The structures were determined by molecular replacement using Phaser (McCoy et al., 2007). Refinement of the structures was carried out using phenix.refine (Adams et al., 2010) and iterations of refinement using Coot (Emsley et al., 2010). Software were accessed through SBGrid (Morin et al., 2013).

### Isothermal Titration Calorimetry

calorimetric titration experiments were performed with an Auto-iTC200 instrument (Malvern) at either 15° C. or 25° C. Proteins were dialyzed against 20 mM Tris pH 8.0 and 150 mM sodium chloride overnight at 4° C. Fabs were concentrated to 10 μM and added to the calorimetric cell, which was titrated with peptide (100 μM) in 15 successive injections of 2.5 μl. Experiments were performed at least twice and the mean and standard error of the mean were reported (FIG. 3). The experimental data were analyzed according to a 1:1 binding model by means of Origin 7.0.

Plasmodium berghei Infections In Vivo

A. gambiae 7b mosquitoes were fed on female CD-1 mice infected with Pb-PfCSP parasites (0.1-0.8% gametocytemia) and kept at 20° C. and 80% humidity until further usage. Infected mosquitoes were offered an additional uninfected blood meal 7 dpi and 20 mosquitoes were dissected for oocyst counts 17 dpi. Female C57BL/6 mice were passively immunized by i.p. injection of 150 or 300 μg of monoclonal antibodies in 200 μl of PBS. After 20 h (18 dpi), mice were exposed to Pb-PfCSP-infected mosquitoes (infection prevalence between 75% and 100%; Supplemental Tables S9-S12). All blood-fed mosquitoes were collected individually for gDNA extraction (NucleoMag VET, Macherey-Nagel) followed by PCR to determine their Pb-PfCSP infectivity status. Specific primers amplifying P. berghei 18s RNA gene (Friesen et al. 2010) and control primers amplifying A. gambiae AGAP001076 gene (Gildenhard et al. 2019) were used. Mosquitoes positive for both PCR reactions were considered infected. Antibody titers were measured by ELISA in serum samples collected from the submandibular vein 2-3 h post mosquito bite. Blood parasitaemia was assayed by daily tests of a minimum of 100 microscopic fields per Giemsa-stained thin blood smears 3-7 days and 10 days post mosquito bite. Infected mice were sacrificed two days after the detection of parasitaemia.

### Statistics

Statistics was performed on Prism 7.04 (GraphPad) or RStudio (version 3.2.2) using two-tailed Mann-Whitney assuming non-normal distribution or Mantel-Cox log-rank test for in vivo experiments, as described in the figure legends. P values less than 0.05 were considered significant (*P<0.05; **P<0.01; ***P<0.001; ****P<0.0001) as indicated in the figure legends.

### Data and Software Availability

The data that support the findings of this report are available from the corresponding authors upon reasonable request. The crystal structures reported herein have been deposited in the Protein Data Bank, www.rcsb.org (PDB ID codes 6O23, 6O24, 6O25, 6O26, 6O28, 6O29, 6O2A, 6O2B, 6O2C).

### EXAMPLE 3: THE MAJORITY OF PFCSP ANTIBODIES ARE CROSS-REACTIVE WITH SEVERAL B CELL EPITOPES

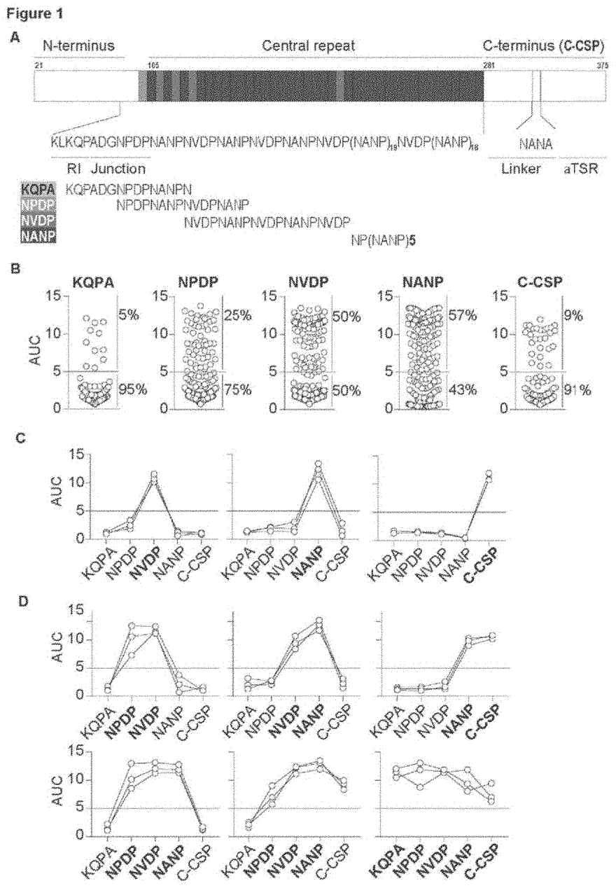

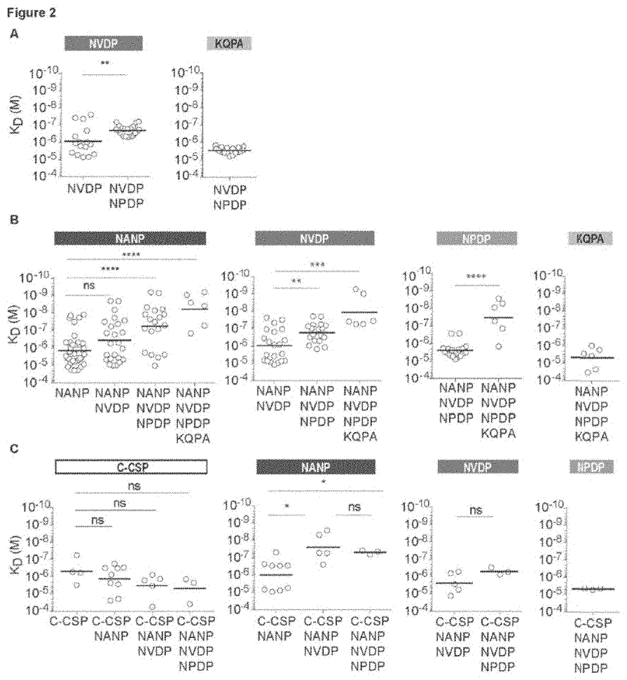

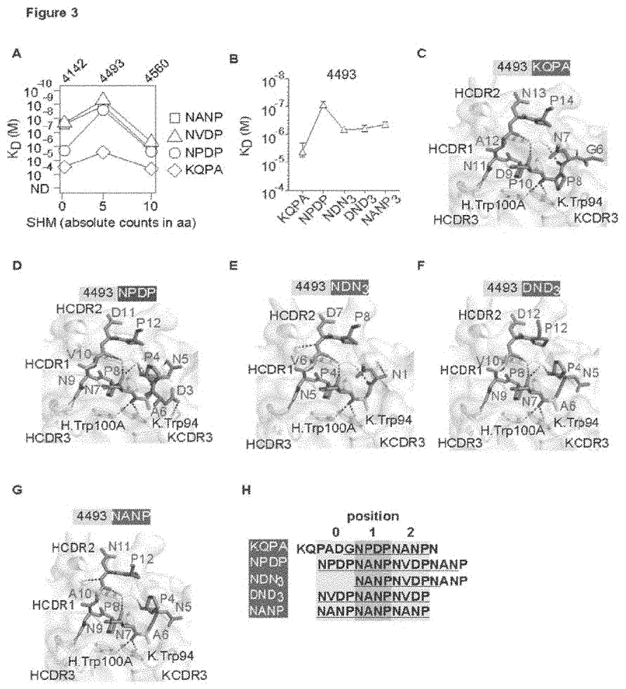

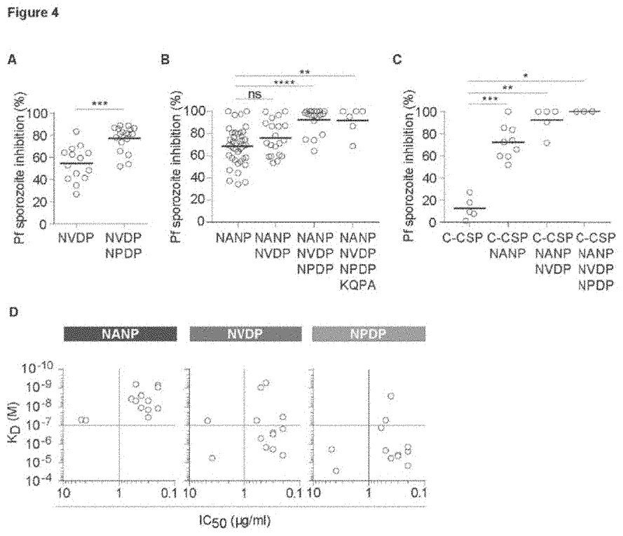

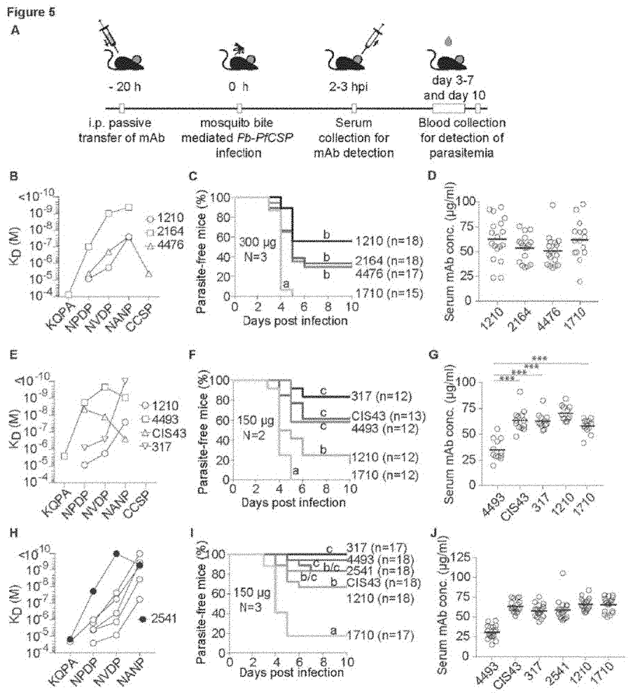

To identify antibodies with reactivity to the different subdomains, we analyzed a large panel of 200 recombinant PfCSP-reactive human monoclonal antibodies (mAbs) for binding to the junctional and C-CSP epitopes by ELISA (FIG. 1). The antibodies were derived from memory B cells of malaria-naïve volunteers after repeated immunization with live sporozoites under chemoprophylaxis (PfSPZ-CVac). Binding was determined to three overlapping peptides in the N-terminal junction containing highly similar NPDP, NVDP, and NANP amino acid (aa) motifs, to a NP(NANP)₅ peptide representative of the central repeat, and to the complete C-terminus (C-CSP) with a unique NANA sequence (FIG. 1A). The N-terminal junction peptides, here abbreviated as KQPA, NPDP, and NVDP according to the first four aa of each peptide, covered aa 95-109 (KQPADGNPDPNANPN, SEQ ID NO:37), aa 101-116 (NPDPNANPNVDPNANP, SEQ ID NO:38), and aa 109-125 (NVDPNANPNVDVNANPNVDP, SEQ ID NO:39), respectively. Nearly 80% of the antibodies (155/200) bound the peptide above our cut-off with an ELISA area under curve (AUC) of >5. The vast majority bound NANP (57%), as previously described (Murugan et al., 2018), and NVDP (50%). Antibodies with reactivity to NPDP (25%) or C-CSP (9%) were less abundant, and only a few recognized KQPA (5%), representing the most N-terminal part of the junction. Several of the 155 antibodies recognized only the NANP repeat (26%), the NVDP peptide (11%), or the C-terminus (3%) but not KQPA, or NPDP and were therefore defined as epitope-specific (FIG. 1C). In contrast, 92/155 antibodies (59%) recognized two or more PfCSP peptides with distinct binding profiles and were therefore referred to as epitope cross-reactive (FIG. 1D). The vast majority (76%) of these cross-binders interacted strongly with the NANP repeat, whereas 24% lacked NANP-reactivity and instead showed preferential binding to NVDP and NPDP. Presumably due to the low degree of sequence similarity between the C terminus and N-terminal junction, C-CSP binding was lower in C-CSP-reactive antibodies with cross-reactivity to the repeat and the N-terminal junction peptides compared to those that only cross-reacted with NANP or compared to C-CSP specific antibodies. The different binding profiles of all antibodies were resolved in a t-distributed stochastic neighbor embedding (t-SNE) analysis by highlighting the ELISA binding strength of each antibody to the individual peptides and to C-CSP. In summary, our analysis identified a high number of PfCSP antibodies with cross-reactivity and a wide spectrum of antigen-binding profiles to the N-terminal junction, the central repeat and C-CSP with highly similar 4-aa NANP, NVDP, NPDP or NANA sequence motifs.

### EXAMPLE 4: CROSS-REACTIVITY WITH THE N-TERMINAL JUNCTION CORRELATES WITH ANTIBODY AFFINITY TO NANP

To delineate the link between cross-reactivity and binding strength, we measured the affinity of antibodies with ELISA-reactivity to the different PfCSP peptides and C-CSP by surface plasmon resonance (SPR; FIG. 2). The data confirmed the high degree of antibody cross-reactivity observed in the ELISA (FIG. 1). Anti-NVDP antibodies with cross-reactivity to the overlapping NPDP peptide showed on average significantly higher NVDP affinities compared to NVDP-specific antibodies (FIG. 2A). Similarly, anti-NANP antibodies with cross-reactivity to the N-terminal junction had higher NANP affinities than NANP-specific antibodies (FIG. 2B). Thus, NANP affinity increased with cross-reactivity to the N-terminal junction and was highest in antibodies that recognized all junctional peptides. The gain in NANP affinity was paralleled by a comparable increase in affinity to NVDP and NPDP. Highest mean affinities to NANP, NVDP and NPDP were observed in cross-reactive antibodies that bound all four peptides, although their KQPA affinity was overall low compared to the other peptides (FIG. 2B). In contrast, strong binding to C-CSP was not associated with cross-reactivity to the repeat and N-terminal junction peptides (FIG. 2C). In summary, high affinity to NANP and NVDP correlated with antibody cross-reactivity to the PfCSP N-terminal junction but not C-CSP.

### EXAMPLE 5: THE CORE PARATOPE OF PFCSP ANTIBODIES PREFERENTIALLY BINDS TO NANP MOTIFS

To understand how a single cross-reactive antibody would bind to the repeat and the N-terminal junction peptides, we investigated mAb 4493, a rare IGHV3-49-, IGKV3-20-encoded antibody that has affinity matured to NANP and the junctional epitopes (FIGS. 3A and 3B). Specifically, we determined the crystal structure of the mAb 4493 Fab fragments in complex with NANP₃ (2.15 Å), two shortened NVDP peptides, NDN₃ (NANPNVDPNANP, SEQ ID NO:40, 2.1 Å) and DND₃ (NVDPNANPNVDP, SEQ ID NO:41, 2.02 Å), NPDP (NPDPNANPNVDPNANP, SEQ ID NO:42, 2.4 Å), and KQPA (KQPADGNPDPNANPN, SEQ ID NO:43, 1.93 Å) (FIG. 3). mAb 4493 contacted all peptides with its HCDR2, HCDR3, and KCDR3. Especially, the IGHV3-49 germline-encoded amino acid H.Arg52 in HCDR2 was strongly involved in direct H-bond contacts with the peptide backbones. The preferential binding of mAb 4493 to NPDP (FIG. 3) correlated with more extensive H-bond interactions compared to the other peptides (11 H-bonds compared to 8 or 9 for DND₃ and NANP₃, respectively), where H.Arg52 alone formed five H-bonds with NPDP.

Strikingly, mAb 4493 bound all peptides in largely superimposable U-shaped conformations (rmsd<0.2 Å) around H.Arg52, similar to IGHV3-33-encoded antibodies, which bound all peptides in inverted S-shape conformations around H.Trp52. The mAb 4493 paratope engaged with three consecutive 4-aa motifs at three distinct positions, here referred to as position 0, 1, and 2. In five of the six structures, position 1 was occupied by NANP illustrating a strong preference of the core paratope for this motif. Indeed, although NPDP could be accommodated at position 1 as observed in the co-complex of mAb 4493 with the KQPA peptide, this binding mode, which placed the NANP motif in position 2, was associated with overall weaker affinity compared to all other peptides including the high-affinity NPDP peptide interaction with NPDP, NANP, and NVDP motifs at positions 0, 1, and 2, respectively (FIG. 3). Compared to the strong preference for NANP motifs at position 1, mAb 4493 showed more flexibility at position 0 as it accommodated NPDP, NANP, and NVDP motifs in the co-complexes with NPDP, NANP₃, and DND₃ peptides, respectively. Binding flexibility was also observed at position 2, which bound NANP and NVDP motifs but not NPDP. Likely due to the strong preference for binding to NANP at position 1, position 2 was more frequently occupied by NVDP, thereby placing NPDP into position 0 according to the natural order of the NPDP-, NANP-, NVDP-motifs in full-length PfCSP. Strikingly, the antigen recognition mode of mAb 4493 was highly similar to CIS43 (Kisalu et al., 2018), a potent cross-reactive IGHV1-2-, IGKV4-1-encoded PfCSP antibody with preference for binding to the N-terminal junction that had been induced by immunization with irradiated sporozoites. Despite differences in the binding orientation between the two antibodies, the peptides bound by mAb 4493 showed highly similar conformations compared to peptides bound by CIS43, which also recognizes recognized junctional peptides through interactions of the core paratope centered on NANP motifs.

### EXAMPLE 6: EVOLUTION OF ANTI-PFCSP ANTIBODY BINDING PROFILES

To determine whether NANP binding played a role in the development of cross-reactive antibodies, we assessed the evolution of the response over time. The frequency of NANP-binding antibodies with strong cross-reactivity to the junctional peptides, especially NPDP and NVDP, increased with repeated parasite exposure and was higher after the third immunization and challenge compared to the second immunization. The vast majority was also class-switched to IgG, including many IGHV3-33-, IGKV1-5-encoded but also rare antibodies with non-prominent gene combinations such as mAb 4493. Most cross-reactive antibodies belonged to clonally expanded and diversified B cell clusters, but the majority showed no differences in their cross-reactivity profile independently of their binding preference and their absolute affinity. To assess what drove the selection of high-affinity cross-reactive antibodies, we compared the kinetic rates of cross-reactive antibodies to associate with (kₒₙ) or dissociate from (k_{off}) NANP, NVDP, and NPDP. Despite similar kₒₙ rates for all peptides, the antibodies differed in their k_{off}, which was significantly lower for binding to the NANP repeat compared to NVDP or NPDP, suggesting that the antibodies had been primarily selected for their ability to bind the repeat and not the junctional peptides. Indeed, although overall infrequent, several of the IGHV3-33- and IGKV1-5-encoded antibodies from PfCSP-reactive memory B cell and also bona-fide plasmablasts carried selected somatic mutations that have been shown to either directly (H.S31, H.V50) or indirectly (H.N56, K.S93) improve NANP binding through homotypic antibody-antibody interactions. Thus in line with our structure analyses, the accumulation of cross-reactive antibodies in response to repeated parasite exposure was likely due to the direct association of antibody cross-reactivity with NANP affinity and continuous selection of high-affinity clones from the naïve repertoire or of B cells that had gained affinity through somatic mutations, especially of IGHV3-33-, IGKV1-5-encoded antibodies.

### EXAMPLE 7: ANTIBODY BINDING TO NANP BUT NOT THE N-TERMINAL JUNCTION OR C-CSP CORRELATES WITH POTENT PF INHIBITION IN VITRO

To determine whether the observed differences in antibody-binding preferences impacted on their Pf-inhibitory capacity, we compared the activity of 139 antibodies with different cross-reactivity profiles to inhibit the sporozoite traversal of hepatocytes in vitro (100 μg/ml). NVDP-specific antibodies were overall poor inhibitors (mean 55%). Although the mean inhibitory activity was significantly higher for NVDP binders with cross-reactivity to NPDP (mean 74%), in line with their higher affinity (FIG. 2), none of the antibodies reached 100% inhibition. NANP-specific antibodies were overall better Pf inhibitors than NVDP-specific antibodies (mean 69%) with two antibodies that conferred complete inhibition (FIG. 4B). The mean potency of NANP-binders increased significantly with cross-reactivity to the N-terminal junction and was higher for antibodies that bound to two or three junctional peptides (mean 92%) than for anti-NANP antibodies with limited cross-reactivity to NVDP only (mean 76%). C-CSP specific antibodies were the weakest inhibitors of all (mean 13%) but their potency was significantly improved with associated cross-reactivity and higher affinity to NANP as previously reported (mean 72%; Scally) and with additional binding to NVDP and NPDP (mean 95%; FIG. 4C). Thus, NANP binding was associated with parasite inhibition, explaining the overall low protective activity of non-NANP-reactive antibodies.

We next examined whether the affinities (K_{D}) of cross-reactive antibodies to NANP, NVDP, or NPDP were predictive of low (50-70%), intermediate (>70-90%) or high (>90%) levels of Pf inhibition (FIG. 4D). NANP, but not NVDP or NPDP affinity was strongly associated with anti-Pf activity. The most potent inhibitors (>90%) were almost exclusively antibodies with NANP affinity below 10⁻⁷ M. The NVDP affinity of the most potent inhibitors was overall weaker (mean K_{D}=1.9×10⁻⁷ M) than their NANP affinity (mean K_{D}=1.7×10⁻⁸ M), and comparable to antibodies with intermediate Pf-inhibitory activity (mean K_{D}=3.4×10⁻⁷ M).

Although rare antibodies with high NPDP affinity were identified in all three groups, NPDP affinity was overall low in the range of K_{D} 10⁻⁶ M and did not discriminate between antibodies with low, intermediate, or high levels of Pf inhibition. In summary, cross-reactive antibodies with low anti-parasite activity were mostly weak binders, whereas high affinity to NANP but not to NVDP or NPDP discriminated the most potent cross-reactive inhibitors from antibodies with intermediate anti-parasite activity.

Next, we selected 15 of the most potent non-clonally-related antibodies with >95% inhibition activity including one NANP-specific and 14 cross-reactive antibodies with different binding profiles and determined their IC₅₀ values. Ten antibodies were encoded by IGHV3-33 in combination with IGKV1-5 (8/10) or IGKV3-20 (2/10), reflecting the strong enrichment of this gene combination among antibodies with >90% Pf inhibitory activity, whereas the other five antibodies were encoded by different IGHV3 and various IGKV and IGLV light chain genes. Regardless of their binding profile, the IC₅₀ values of all 15 antibodies ranged from 0.2 μg/ml to >20 μg/ml and correlated with their NANP (K_{D} 10⁻⁷-10⁻⁹ M) but not their NVDP (K_{D} 10⁻⁶-10⁻⁹ M) or NPDP (K_{D} 10⁻⁵-10⁻⁹ M) affinities (FIG. 4D). Strikingly, with one exception, the IC₅₀ values of all ten IGHV3-33-encoded antibodies were <1.0 μg/ml (mean=0.68 μg/ml). In contrast, only 2/5 antibodies with other gene combinations showed IC₅₀ values <1.0 μg/ml, whereas for the other three, the values ranged from 4.7 μg/ml to >20 μg/ml. Thus, non-IGHV3-33-, encoded antibodies with high Pf-inhibitory activity were rare. The most potent antibodies in this assay all showed NANP affinities <10⁻⁷ M independently of their binding profiles and included cross-reactive and rare epitope-specific antibodies.

### EXAMPLE 8: ANTIBODY-MEDIATED PROTECTION FROM THE INFECTION IN VIVO

To determine the inhibitory activity of the most potent antibodies in vivo, we measured the protective efficacy of the antibodies as time to the development of blood-stage parasites (prepatency) in mice infected with PJCSP-expressing transgenic Plasmodium berghei parasites (Pb-PfCSP) by mosquito bites 20 h after intraperitoneal antibody injection (300 μg/mouse, FIG. 5A). First, we assessed the potency of three cross-reactive IGHV3-33-encoded antibodies with IC₅₀ values <1.0 μg/ml (mAbs 1210, 2164, 4476) compared to a non-protective C-CSP-specific negative control (mAb 1710, Scally). mAbs 4476 and 1210 had comparable NANP, NVDP, and NPDP affinities, but only mAb 4476 recognized C-CSP. In contrast, mAb 2164 bound NANP, NVDP, and NPDP with up to 2,000-fold higher affinity and also cross-reacted with KQPA but not C-CSP. The highest protection (56% parasitemia-free mice) was observed for mAb 1210, but the differences compared to mAb 2164 (33%) and mAb 4476 (30%) were not statistically significant (Table S8). Thus, affinity and cross-reactivity with the N-terminal junction or C-CSP did not predict the in vivo potency of these IGHV3-33-encoded antibodies.

To determine whether the same was true for non-IGHV3-33-encoded antibodies, we compared the potency of mAb 4493 (IGHV3-49-, IGKV3-20) with mAb 1210 in the same model (FIGS. 5E-5G). Additionally, we included two potent published non-IGHV3-33-encoded antibodies. mAb CIS43 (IGHV1-3, IGKV4-1; Kisalu et al., 2018) was similar to mAb 4493 in its strong affinity for NPDP, but showed no measurable KQPA reactivity and about 10-fold and 100-fold lower NVDP and NANP affinity, respectively. mAb 317 (IGHV3-30-3, IGKV1-5; Oyen et al., 2017) had an exceptionally high NANP affinity (<10⁻¹⁰ M) with relatively low cross-reactivity to NVDP and NPDP (FIG. 5E). At a dose of 300 μg per mouse, mAbs 4493, CIS43, and 317 showed similar levels of protection from blood-stage parasitemia of 83%, 91%, and 100%, respectively. To better resolve potential differences in the inhibitory activities of the antibodies, we halved the dose to 150 μg per mouse (FIGS. 5F and 5G). Although the degree of protection was overall lower, all three antibodies retained their high potency compared to mAb 1210. mAb 317 protected 83% of mice compared to 62% for mAb CIS43 and 58% for mAb 4493, but these differences were not statistically significant. Of note, independent of the dose, mAb 4493 consistently showed on average two-fold lower serum concentrations at the time of challenge than the other antibodies (FIG. 5G).

To determine whether IGHV3-33-encoded antibodies could reach similar levels of potency, we compared the antibodies to mAb 2541, a high affinity cross-reactive IGHV3-33-, IGKV1-5-encoded plasmablast antibody with 8-aa-long KCDR3 and an IC₅₀<1 μg/ml in the in vitro Pf-traversal inhibition assay. Notably, mAb 2541 showed higher affinity to NANP and NVDP than any IGHV3-33-, IGKV1-5-encoded memory B cell antibody in our panel, as well as additional cross-reactivity to NPDP and KQPA (FIG. 5H). In direct comparison to mAbs CIS43, 4493, and 317, mAb 2541 was as protective as these non-IGHV3-33-encoded antibodies (FIGS. 51 and 5J). Thus, the most potent PfCSP antibodies with high levels of in vivo protection against malaria parasites showed exceptional affinity to the repeat or the junctional epitopes and were encoded by IGHV3-33 (mAb 2541) or other gene combinations (mAbs 4493).

### LITERATURE CITED

Adams et al., 2010, Acta Crystallogr. Sect. D Biol. Crystallogr. 66, 213-221
Emsley et al., 2010, Acta Crystallogr. D Biol. Crystallogr. 66, 486-501
Friesen et al. 2010, Science Translational Medicine; 2(40):40ra49
Gildenhard et al. 2019, Nat Microbiol. 4(6):941-947
Kabsch et al., 2010, Acta Crystallogr. D Biol. Crystallogr. 66, 125-132
Kisalu et al., 2018, Nat. Med. 24, 408-416
McCoy et al., 2007, J. Appl. Cryst. 40, 658-674
Mordmüller et al., 2017, Nature; 542(7642):445-449
Morin et al., 2013, eLife 2, e01456
Murugan et al., 2018, Sci. Immunol. 3, eaap8029
Oyen et al., 2017, Proc. Natl. Acad. Sci. U.S.A 114, E10438-E10445
Pompon and Levashina, 2015, PLoS Biol. 13(9):e1002255
Sattabongkot et al., 2006, Am J Trop Med Hyg., 74(5):708-15
Tan, et al., Nat. Med. 24, 401-407
Tiller et al. 2008, J Immunol. Methods 329:112-124
Tiller et al., 2009, J Immunol Methods. 350:183-193
Triller et al., Immunity 47, 1197-1209.e10 (2017)
Wardemann et al., 2003, Science 301:1374-1377

1. An antibody binding to a peptide comprising an amino acid sequence NANP (SEQ ID NO:1) and to at least one peptide comprising an amino acid sequence selected from NVDP (SEQ ID NO:2), NPDP (SEQ ID NO:3), and KQPA (SEQ ID NO:4), wherein the peptide comprising the amino acid sequence NANP consists of the amino acid sequence NPNANPNANPNANPNANPNANP (SEQ ID NO:44) or NANPNANPNANPNANPNANP (SEQ ID NO:45), wherein the peptide comprising the amino acid sequence KQPA consists of the amino acid sequence KQPADGNPDPNANPN (SEQ ID NO:37), wherein the peptide comprising the amino acid sequence NPDP consists of the amino acid sequence NPDPNANPNVDPNANP (SEQ ID NO:38), and wherein the peptide comprising the amino acid sequence NVDP consists of the amino acid sequence NVDPNANPNVDPNANPNVDP (SEQ ID NO:39).

2. The antibody of claim 1, wherein the dissociation constant K_{D} for the antibody and the peptide comprising an amino acid sequence NANP is at most 10⁻⁶ M, preferably at most 2×10⁻⁷ M, more preferably at most 10⁻⁷ M, even more preferably at most 5×10⁻⁸ M, still more preferably at most 2×10⁻⁸ M, most preferably at most 10⁻⁸ M, wherein the dissociation constant K_{D} for the antibody and the peptide comprising an amino acid sequence NVDP or NPDP is at most 10⁻⁶ M, preferably at most 2×10⁻⁷ M, more preferably at most 10⁻⁷ M, even more preferably at most 5×10⁻⁸ M, still more preferably at most 2×10⁻⁸ M, most preferably at most 10⁻⁸ M, and/or wherein the dissociation constant K_{D} for the antibody and the peptide comprising an amino acid sequence KQPA is at most 10⁻⁵ M, preferably at most 5×10⁻⁶ M, more preferably at most 2×10⁻⁶ M, most preferably at most 10⁻⁷ M.

3. The antibody of claim 1, wherein said antibody comprises complementarity determining regions (CDRs) comprising the sequences of SEQ ID NOs:5 to 10 or SEQ ID NOs:11 to 16 or comprises complementarity determining regions (CDRs) comprising sequences at least 80% identical to the sequences of SEQ ID NOs:5 to 10 or SEQ ID NOs:11 to 16.

4. The antibody of claim 1, wherein said antibody comprises
(i) an amino acid sequence of the heavy chain as shown in SEQ ID NO:29 or a sequence at least 50% identical to SEQ ID NO:29; and an amino acid sequence of the light chain as shown in SEQ ID NO:30 or a sequence at least 50% identical to SEQ ID NO:30; or
(ii) an amino acid sequence of the heavy chain as shown in SEQ ID NO:31 or a sequence at least 50% identical to SEQ ID NO:31; and an amino acid sequence of the light chain as shown in SEQ ID NO:32 or a sequence at least 50% identical to SEQ ID NO:32.

5. The antibody of claim 1, wherein said antibody is a monclonal antibody or a fragment thereof; and/or is a human or a humanized antibody.

6. (canceled)

7. (canceled)

8. A method of preventing a Plasmodium infection in a subject, comprising contacting said subject with the antibody according to claim 1.

9. A method for detecting a Plasmodium circumsporozoite protein in a sample, comprising
a) contacting said sample with an antibody according to any one of claims 1 to 5, and thereby
b) detecting said Plasmodium circumsporozoite protein in said sample.

10. The method of claim 9, wherein said method comprises further step al) detecting binding of said antibody to said Plasmodium circumsporozoite protein.

11. A method for detecting an antibody suitable for preventing malaria, comprising
a) providing a candidate antibody suspected to be suitable for preventing malaria;
b) determining an affinity of said candidate antibody to a peptide comprising the amino acid sequence NANP (SEQ ID NO:1);
c) determining an affinity of said candidate antibody to a peptide comprising at least one amino acid sequence selected from NVDP (SEQ ID NO:2), NPDP (SEQ ID NO:3), and KQPA (SEQ ID NO:4); and
d) identifying an antibody suitable for preventing malaria based on the results of steps b) and c).

12. The method of claim 11, wherein an antibody suitable for preventing malaria is identified if (i) it is determined in step b) that the dissociation constant K_{D} for the candidate antibody and the peptide comprising an amino acid sequence NANP is at most 10⁻⁶ M, preferably at most 2×10⁻⁷ M, more preferably at most 10⁻⁷ M, even more preferably at most 5×10⁻⁸M, still more preferably at most 2×10⁻⁸M, most preferably at most 10⁻⁸ M; and (ii) it is determined in step c) that the dissociation constant K_{D} for the candidate antibody and the peptide comprising an amino acid sequence NVDP, NPDP, and KQPA is at most 10⁻⁶ M, preferably at most 2×10⁻⁷ M, more preferably at most 10⁻⁷ M, even more preferably at most 5×10⁻⁸ M, still more preferably at most 2×10⁻⁸ M, most preferably at most 10⁻⁸ M and/or the dissociation constant K_{D} for the candidate antibody and the peptide comprising an amino acid sequence KQPA is at most 10⁻⁵ M, preferably at most 5×10⁻⁶ M, more preferably at most 2×10⁻⁶ M, most preferably at most 10⁻⁷ M.

13. (canceled)

14. (canceled)

15. (canceled)

16. (canceled)

17. (canceled)

18. The method of claim 8, wherein said plasmodium infection is a Plasmodium falciparum infection.

19. The method of claim 8, wherein said preventing a Plasmodium infection comprises preventing malaria.


SEQUENCE LISTING (46 sequences)

SEQ ID NO: 1 - protein, 4 aa, Plasmodium falciparum
NANP

SEQ ID NO: 2 - protein, 4 aa, Plasmodium falciparum
NVDP

SEQ ID NO: 3 - protein, 4 aa, Plasmodium falciparum
NPDP

SEQ ID NO: 4 - protein, 4 aa, Plasmodium falciparum
KQPA

SEQ ID NO: 5 - protein, 8 aa, Homo sapiens
GFTFGDYA

SEQ ID NO: 6 - protein, 10 aa, Homo sapiens
IRSKANGGRT

SEQ ID NO: 7 - protein, 13 aa, Homo sapiens
TRVELGSSWSLGY

SEQ ID NO: 8 - protein, 7 aa, Homo sapiens
QSVSSTY

SEQ ID NO: 9 - protein, 3 aa, Homo sapiens
GAS

SEQ ID NO: 10 - protein, 9 aa, Homo sapiens
QQYGSSPWT

SEQ ID NO: 11 - protein, 8 aa, Homo sapiens
GFTFSSYG

SEQ ID NO: 12 - protein, 8 aa, Homo sapiens
IWHDGSKK

SEQ ID NO: 13 - protein, 16 aa, Homo sapiens
ARVGDYSDFKYGAFDI

SEQ ID NO: 14 - protein, 6 aa, Homo sapiens
QSISSW

SEQ ID NO: 15 - protein, 3 aa, Homo sapiens
KAS

SEQ ID NO: 16 - protein, 8 aa, Homo sapiens
QQYNSYWT

SEQ ID NO: 17 - DNA, 24 bp, Homo sapiens
ggattcacctttggtgattatgct

SEQ ID NO: 18 - DNA, 30 bp, Homo sapiens
attagaagcaaagctaatggtgggagaaca

SEQ ID NO: 19 - DNA, 39 bp, Homo sapiens
actagagttgaactaggcagcagctggtcccttggttac

SEQ ID NO: 20 - DNA, 21 bp, Homo sapiens
cagagtgttagcagcacctac

SEQ ID NO: 21 - DNA, 9 bp, Homo sapiens
ggtgcatcc

SEQ ID NO: 22 - DNA, 27 bp, Homo sapiens
cagcagtatggtagctcaccgtggacg

SEQ ID NO: 23 - DNA, 24 bp, Homo sapiens
ggattcaccttcagcagttatggc

SEQ ID NO: 24 - DNA, 24 bp, Homo sapiens
atatggcatgatggaagtaaaaaa

SEQ ID NO: 25 - DNA, 48 bp, Homo sapiens
gcgagggtgggggactacagtgacttcaaatacggtgcttttgatatc

SEQ ID NO: 26 - DNA, 18 bp, Homo sapiens
cagagtattagtagttgg

SEQ ID NO: 27 - DNA, 9 bp, Homo sapiens
aaggcgtct

SEQ ID NO: 28 - DNA, 24 bp, Homo sapiens
caacaatataatagttattggacg

SEQ ID NO: 29 - protein, 122 aa, Homo sapiens
EVQLVESGGGLVQPGRSLRLSCTASGFTFGDYAMSWVRQAPGKGLEWVGFIRSKANGGRTENPASVKGRFTISRDDSKSIAYLQMNSLKTEDTAVYYCTRVELGSSWSLGYWGQGTLVTVSS

SEQ ID NO: 30 - protein, 108 aa, Homo sapiens
EIVLTQSPGTLSLSPGERATLSCRASQSVSSTYLAWYQQKPGQAPRLLIYGASSRATGIPDRFSGSGSGTDFTLTISRLEPEDFAVYYCQQYGSSPWTFGQGTKVEIK

SEQ ID NO: 31 - protein, 123 aa, Homo sapiens
QVQLVESGGGVVQPGRSLRLSCAASGFTFSSYGMHWVRQTTGKGLEWVAIIWHDGSKKYYADSVKGRLTISRDNSKNTLYLQMNSLRVEDTALYYCARVGDYSDFKYGAFDIWGQGTMVTVSS

SEQ ID NO: 32 - protein, 106 aa, Homo sapiens
DIQMTQSPSTLSASVGDRVTITCRASQSISSWLAWYQQKPGKAPNLLIYKASTLESGVPSRFSGSGSGTEFTLTISSLQPDDFATYYCQQYNSYWTFGQGTRVEIK

SEQ ID NO: 33 - DNA, 367 bp, Homo sapiens
gaagtgcagctggtggagtctgggggaggcttggtacagccagggcggtccctgagactctcctgtacagcttctggattcacctttggtgattatgctatgagctgggtccgccaggctccagggaaggggctggagtgggtaggtttcattagaagcaaagctaatggtgggagaacagaaaaccccgcgtccgtgaaaggcagattcaccatctcaagagatgattccaaaagcatcgcctatctgcaaatgaacagcctgaaaaccgaggacacagccgtgtattactgtactagagttgaactaggcagcagctggtcccttggttactggggccagggaaccctggtcaccgtctcctcag

SEQ ID NO: 34 - DNA, 325 bp, Homo sapiens
gaaattgtgttgacacagtctccaggcaccctgtctttgtctccaggggaaagagccaccctctcctgcagggccagtcagagtgttagcagcacctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgtggacgttcggccaagggaccaaggtggagatcaaac

SEQ ID NO: 35 - DNA, 370 bp, Homo sapiens
caggtgcagctggtggagtcggggggaggcgtggtccagcctgggaggtccctgagactctcctgtgcagcgtctggattcaccttcagcagttatggcatgcactgggtccgccagactacaggcaaggggctggagtgggtggcgattatatggcatgatggaagtaaaaaatattatgcagactccgtgaagggccgactcaccatctccagagacaattccaagaacacgctgtatctgcaaatgaacagcctgagagtcgaggacacggctctttattactgcgcgagggtgggggactacagtgacttcaaatacggtgcttttgatatctggggccaagggacaatggtcaccgtctcttcag

SEQ ID NO: 36 - DNA, 319 bp, Homo sapiens
gacatccagatgacccagtctccttccaccctgtctgcatctgtaggagacagagtcaccatcacttgccgggccagtcagagtattagtagttggttggcctggtatcagcagaaaccagggaaagcccctaacctcctgatctataaggcgtctactttagaaagtggggtcccatcaaggttcagcggcagtggatctgggacagaattcactctcaccatcagcagcctgcagcctgatgattttgcaacttattactgccaacaatataatagttattggacgttcggccaagggaccagggtggaaatcaaac

SEQ ID NO: 37 - protein, 15 aa, Plasmodium falciparum
KQPADGNPDPNANPN

SEQ ID NO: 38 - protein, 16 aa, Plasmodium falciparum
NPDPNANPNVDPNANP

SEQ ID NO: 39 - protein, 20 aa, Plasmodium falciparum
NVDPNANPNVDVNANPNVDP

SEQ ID NO: 40 - protein, 12 aa, Artificial Sequence
  NDN3 peptide
NANPNVDPNANP

SEQ ID NO: 41 - protein, 12 aa, Artificial Sequence
  DND3 peptide
NVDPNANPNVDP

SEQ ID NO: 42 - protein, 16 aa, Artificial Sequence
  NPDP
NPDPNANPNVDPNANP

SEQ ID NO: 43 - protein, 15 aa, Artificial Sequence
  KQPA
KQPADGNPDPNANPN

SEQ ID NO: 44 - protein, 22 aa, Artificial Sequence
  NANP peptide1
NPNANPNANPNANPNANPNANP

SEQ ID NO: 45 - protein, 20 aa, Artificial Sequence
  NANP peptide2
NANPNANPNANPNANPNANP

SEQ ID NO: 46 - protein, 188 aa, Plasmodium falciparum
KLKQPADGNPDPNANPNVDPNANPNVDPNANPNVDPNANPNANPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANP